(19) United States
(12) Patent Application Publication
(10) Pub. No.: US 2007/0106143 A1
(43) Pub. Date: May 10, 2007

(54) Electrode arrays and related methods

(76) Inventor: J. Christopher Flaherty, Topsfield, MA (US)

Correspondence Address: Leslie I. Bookoff;FINNEGAN, HENDERSON, FARABOW,, GARRETT & DUNNER, L.L.P., 901 New York Avenue, N.W., Washington, DC, 20001-4413, US

(21) Appl. No.: 11/268,637

(22) Filed: Nov. 8, 2005

(51) Int. Cl. A61B 5/04; A61N 1/00

(52) U.S. Cl. 600/373; 600/393; 607/116

(57) ABSTRACT

Various embodiments of an electrode array system and related methods are disclosed. The system may include a probe assembly having a plurality of probes configured to penetrate tissue of a patient and a guide assembly having a plurality of guiding channels. Each of the guiding channels may be configured to guide one or more of the plurality of probes to a desired tissue site. Some embodiments of an electrode array may include a housing and a plurality of probes extending from the housing. At least one of the plurality of probes may be individually deployable from the housing.

### FIELD OF THE INVENTION

Embodiments of the present invention relate generally to electrode array systems and related methods. More specifically, particular embodiments of the invention relate to electrode array systems having a guide assembly and/or a probe deploying mechanism for placement in, for example, a patient's body.

### DESCRIPTION OF RELATED ART

Various parts of the body, such as, for example, sensory organs, may generate signals (e.g., electrical signals) and transmit them to the brain. The brain receives these signals and in turn generates suitable electrical signals to control movements of various body parts. To provide access to these electrical signals associated with numerous types of living cells in the patient's body, certain devices, such as those including one or more sensors, may be implanted in various locations within the patient's body.

Recent advances in neurophysiology have allowed researchers to detect and study the electrical activity of highly localized groups of neurons in the brain and/or other nerve tissues in various body parts with high temporal accuracy. The information in the sensed electrical activity may include a variety of information, including physiologic information, sensory information, and motor mapping information. These advances have created the possibility of extracting and processing that information and creating brain-machine interfaces (BMIs) that may allow, for example, treatment of certain neurological disorders and restoration of lost function caused by traumatic injury. For example, with one or more sensors (e.g., electrode arrays) implanted in the higher brain regions that control voluntary movement, signals generated by the patient while imagining such movement may be detected by the sensors. The sensor then may generate electrical signals that can be processed by a suitable signal processing unit to create thought-invoked control signals. Such control signals may be used to control numerous devices including, but not limited to, computers, communication devices, external prostheses (e.g., artificial arm or leg), robots, and other various remote control devices.

Various sensors have been used to detect electrical activity in the brain. For example, noninvasive sensors, such as multi-channel electroencephalogram (EEG) sensors placed on the surface of a patient's scalp, have been used as simple BMIs or otherwise to record brain activity. EEG sensors, however, may not offer sufficient temporal or spatial resolution needed for various applications including, for example, prosthetic controls, detecting single cell activity, or fine graining a seizure focus. Instead, EEG sensors detect mass fluctuations of averaged neuron activity and, therefore, provide much simpler, reduced forms of neuron activity information without providing information about the activity of single cells or their interactions.

Thus, current research into the electrical activity of single cells or small groups of neural cells has been performed primarily with arrays of microelectrodes inserted into the brain. These microelectrode systems may be classified into two broad groups: those having microdrive mechanisms and those having fixed electrode arrays. Systems with microdrive mechanisms may allow a single electrode to be vertically positioned with respect to the brain tissue and allow the electrode to be individually driven by the microdrive mechanism. Thus, a user may actively search for neurons of interest and accurately position the electrode tip near the soma of the neuron to improve the signal-to-noise ratio. Such systems, however, may not be fully implanted in a human because individual microdrive mechanisms are relatively bulky. Moreover, microdrive systems typically cannot use more than a few dozen electrodes due to space limitations and the time it takes to independently position each electrode near a neuron.

Fixed electrode array systems overcome some of these problems, but have their own problems as well. Since the electrodes are fixed, once placed in the brain, the electrodes may not be repositioned, depriving the ability to actively search for neurons. Moreover, these electrode assemblies are typically straight and relatively rigid and, therefore, may not be suitable to be positioned on a surface having a non-flat configuration (e.g., a surface having crevices or sulcus). Wire bundle electrode assemblies, which are difficult to place accurately, have similar disadvantages.

Accordingly, there is a need for an improved electrode array system that may overcome one or more of the problems discussed above. In particular, there is a need to develop a multi-probe, multi-electrode system, where individual electrodes may be capable of being accurately positioned in a broad range of desired tissue sites.

### SUMMARY OF THE INVENTION

Therefore, various exemplary embodiments of the invention may provide electrode array systems having a guide assembly configured to guide individual probes carrying the electrodes to the desired tissue sites and/or a probe deploying mechanism configured to separately deploy individual probes carrying the electrodes so as to enable a user to actively search for signal-generating tissue of interest and accurately position the electrode tip to the desired tissue site.

To attain the advantages and in accordance with the purpose of the invention, as embodied and broadly described herein, one exemplary aspect of the invention may provide an electrode system comprising a probe assembly having a plurality of probes configured to penetrate tissue of a patent and a guide assembly having a plurality of guiding channels. Each of the guiding channels may be configured to guide one or more of the plurality of probes to a desired tissue site.

In one exemplary aspect, at least one of the plurality of probes may be configured to detect cellular signals. In another aspect, at least one of the plurality of probes may be configured to deliver energy to tissue. The energy delivered to tissue may be selected from the group consisting of: heat energy; cryogenic energy; light energy; radiation energy; chemical energy; mechanical energy; electrical energy; and any combination thereof.

According to another exemplary aspect, at least one of the probes may be configured to deliver agent. The agent may comprise a pharmaceutical agent. In still another exemplary aspect, at least one of the probes may be configured to produce a magnetic field.

In yet still another exemplary aspect, at least one of the probes may comprise a sensor. The sensor may be selected from the group consisting of: a thermal sensor; a pressure sensor; a chemical sensor; a force sensor; an electromagnetic field sensor; a physiologic sensor; a photodetector; a pH sensor; an oxygen sensor; a blood sensor; an electrode; and any combination thereof. The physiologic sensor may comprise at least one of an electrocardiogram sensor and a blood glucose sensor.

According to another aspect, at least one of the probes may be flexible. Alternatively or additionally, at least one of the probes is rigid. In some aspects, at least one of the probes may have a resiliently biased shape. The resiliently biased shape may have a curved portion.

In one aspect, at least one of the probes may comprise a shape memory material. The shape memory material may comprise a shape memory alloy. In another exemplary aspect, at least two of the probes may have lengths that are different from one another. Alternatively or additionally, at least two of the probes may have thicknesses that are different from one another.

In some exemplary aspects, at least one of the probes may comprise a first functional element and a second functional element. At least one of the first and second functional elements may comprise an electrode. The electrode may be located at a distal tip of the probe.

In another aspect, the first functional element may be an electrode with a first set of characteristics, and the second functional element may be an electrode with a second set of characteristics. The characteristics may comprise at least one of: an impedance; a surface area; a material of construction; a surface texture; a porosity; a length; a width; a diameter; a thickness; a surface energy; a coating; and any combination thereof. In still another aspect, at least one of the first and second functional elements may comprise at least one of a photodiode and a photosensor.

According to still another exemplary aspect, at least one of the probes or at least one of the guiding channels may comprise a conductive trace. The conductive trace may be configured to provide an electrical connection between the at least one of the probes and the at least one of the guiding channels.

In one exemplary aspect, at least one of the probes may comprise a lumen along at least a portion of its length. The lumen may be configured to permit passage of fluid.

According to another exemplary aspect, the plurality of probes may be arranged in an array. In some exemplary aspects, the probe assembly may comprise a housing from which the plurality of probes may project, and at least one of the plurality of probes may be individually deployable from the housing. In an aspect, the housing may comprise a probe deployment mechanism configured to move the at least one of the plurality of probes relative to the housing. According to another aspect, the housing may comprise at least one internal guiding lumen configured to receive one or more probes of the probe assembly. The housing may comprise a drive assembly positioned adjacent the internal guiding lumen to move the one or more probes within the internal guiding lumen.

In another exemplar aspect, at least one of the probes may be configured to be deployed while the housing is being implanted on the tissue of the patient or after the housing is implanted on the tissue of the patient. For example, at least one probe may be configured to be deployed during or after the implantation of the system (e.g., more than 30 days after the implantation).

In another exemplary aspect, the housing may comprise at least one internal guiding lumen configured to receive at least one probe of the probe assembly, and the housing may comprise a drive assembly positioned adjacent the internal guiding lumen to move the probe within the internal guiding lumen.

According to one exemplary aspect, the drive assembly may be controllable manually. Alternatively or additionally, the drive assembly may be controllable remotely. Moreover, the drive assembly may be controllable automatically.

In another exemplary aspect, the drive assembly may comprise a screw extending along at least a portion of the internal guiding lumen, a drive member configured to engage the probe and the screw, and a drive mechanism configured to drive the drive member so as to move the probe along the internal guiding lumen.

In still another exemplary aspect, the drive assembly may comprise at least one pinch roller in contact with the probe, wherein rotating the roller may cause the probe to move distally or proximally along the internal guiding lumen. At least one pinch roller may be disposed adjacent the internal guiding lumen or has a portion disposed in the internal guiding lumen. In some aspects, the at least one pinch roller may comprise two pinch rollers.

In yet still another exemplary aspect, the drive assembly may comprise a gas discharging member having an outlet valve and being configured to discharge gas into the internal guiding lumen, and a gas suction member having an inlet valve and being configured to suction gas out of the internal guiding lumen. Discharge of the gas may cause the probe to advance the probe distally along the internal guiding lumen, and suctioning of the gas may cause the probe to retract proximally along the internal guiding lumen. In one exemplary aspect, the gas discharging member may comprise an electrolytic cell.

According to another exemplary aspect, the drive assembly may comprise an extendable piston having a distal end connected to the probe, and a drive assembly configured to extend or retract the extendable piston so as to move the probe distally or proximally along the internal guiding lumen. In an exemplary aspect, the drive mechanism may comprise at least one of a hydraulic drive element and a pneumatic drive element.

In one exemplary aspect, the drive assembly may comprise a roller coupled to a proximal end of the probe, where a surface of the roller may be in contact with an inner surface of the internal guiding lumen, and a controller configured to control rotation of the roller. Rotation of the roller may cause the probe to move distally or proximally along the internal guiding lumen. In another exemplary aspect, the drive assembly may comprise a second roller coupled to the probe.

According to another exemplary aspect, the drive assembly may comprise a tube having inner threads and being disposed inside the internal guiding lumen, a screw attached to a proximal end of the probe, where the screw may be configured to engage with and ride over the inner threads, and a drive mechanism configured to rotate at least one of the tube and the screw. Rotating at least one of the tube and the screw may cause the screw to move relative to the tube so as to move the probe distally or proximally along the internal guiding lumen. In one exemplary aspect, the drive mechanism may comprise a stepper motor

In still another exemplary aspect, the drive assembly may comprise first teeth disposed at least partially along the internal guiding lumen, a drive member coupled to the probe, the drive member comprising a forward-moving member configured to engage or disengage at least one tooth of the first teeth when a first predetermined condition is applied, such that when the forward-moving member engages and disengages the at least one tooth of the first teeth, the probe moves distally along the internal guiding lumen, and an actuator configured to actuate the forward-moving member so as to move the probe distally along the internal guiding lumen.

In another exemplary aspect, the forward-moving member may comprise a shape memory material. According to still another aspect, the forward-moving member may be disengaged from the at least one tooth of the first teeth when the probe moves proximally.

In still another exemplary aspect, the drive assembly may further comprise second teeth disposed at least partially along the internal guiding lumen. The drive member may further comprise a backward-moving member configured to engage or disengage at least one tooth of the second teeth when a second predetermined condition is applied, so that when the backward-moving member engages and disengages the at least one tooth of the second teeth, the probe moves proximally along the internal guiding lumen.

According to one exemplary aspect, the drive assembly may comprise teeth disposed at least partially along the internal guiding lumen, a drive member coupled to the probe, the drive member comprising a backward-moving member configured to engage or disengage at least one tooth when a first predetermined condition is applied, such that when the backward-moving member engages and disengages the at least one tooth of the teeth, the probe moves proximally along the internal guiding lumen, and an actuator configured to actuate the backward-moving member so as to move the probe proximally along the internal guiding lumen.

In another exemplary aspect, the backward-moving member may comprise a shape memory material. In still another exemplary aspect, the backward-moving member may be disengaged from the at least one tooth when the probe moves distally.

In yet still another exemplary aspect, the drive assembly may comprise at least one first magnet disposed at least partially along the internal guiding lumen, a drive member coupled to the probe and comprising at least one second magnet, and a controller for energizing either the first magnet or the second magnet. Energizing either the first magnet or the second magnet may cause the probe to move distally or proximally along the internal guiding lumen. In one aspect, at least one of the first and second magnets may be configured to be activated by the controller. In another aspect, at least one of the first and second magnets may be an electromagnet. Alternatively or additionally, at least one of the first and second magnets may be a permanent magnet.

According to some exemplary aspects, one of the first and second magnets may be an electromagnet, and the other of the first and second magnets may be a permanent magnet. In one exemplary aspect, at least one of the first and second magnets may comprise a plurality of magnets.

In another aspect, the controller may be configured to energize either the first magnet or the second magnet by supplying electrical current to the magnet to be energized. The electrical current supplied in a first direction may cause the probe to move distally along the internal guiding lumen, and the electrical current supplied in a second direction opposite the first direction may cause the probe to move proximally along the internal guiding lumen.

According to still another exemplary aspect, the at least one first magnet or the at least one second magnet may comprise a plurality of magnets arranged in a row, and the plurality of magnets may be separated from one another by a predetermined distance. Energizing either the first magnet or the second magnet may cause the probe to move a length that is substantially equal to the predetermined distance.

According to another exemplary aspect, the housing may further comprise at least one of: a memory storage device; a signal processing unit; a power transfer device; a power conversion device; a wireless communication device; a CPU; a microcontroller; a drug delivery assembly or reservoir; an electromagnetic field generator; a light source; a camera assembly; an impedance measurement device; a radiopaque marker; and a power supply.

In still another exemplary aspect, the housing may further comprise a power transfer device configured to convert non-electrical energy to electrical energy. In yet still another exemplary aspect, the housing may comprise a wireless communication device configured to transfer information via: radiofrequency; microwave; infrared; ultrasound; or any combination thereof.

In some exemplary aspects, the housing may further comprises a signal processing element configured to perform a signal processing function selected from the group consisting of: amplification; filtering; sorting; conditioning; translating; interpreting; encoding; decoding; combining; extracting; sampling; multiplexing; analog to digital converting; digital to analog converting; mathematically transforming; and any combination thereof.

In one aspect, the guide assembly may comprise a tissue contacting surface, and at least one of the guiding channels may comprise a portion that may be substantially parallel to at least a portion of the tissue contacting surface. In another aspect, the guide assembly may comprise a tissue contacting surface, and at least one of the guiding channels may comprise a portion that forms an approximately 45° angle with respect to at least a portion of the tissue contacting surface.

In still another exemplary aspect, at least one of the guiding channels or the corresponding one or more probes received in the at least one of the guiding channels may comprise a conductive trace configured to provide an electrical connection between the at least one of the guiding channels and the one or more probes. Energy and/or signals may be transferred via the electrical connection.

According to some exemplary aspects, at least one of the plurality of probes may comprise one or more reservoirs or ports for delivery of an agent. In some exemplary embodiments, the agent may be a fluid. The probe assembly may comprise a pump configured to supply the agent to the one or more reservoirs or ports. At least one of the one or more reservoirs or ports may be refillable.

In one exemplary aspect, the probe assembly may be a micro electro-mechanical system. The micro electromechanical system may be integrated into a silicon substrate.

In another exemplary aspect, at least one of the plurality of probes may comprise at least one electrode. In still another exemplary aspect, at least one of the plurality of probes may comprise at least one of: a recording electrode; a stimulating electrode; a sensor; an acoustic transducer; a light source; a heat source; a cooling source; an agent eluding port; and a reservoir.

According to still another exemplary aspect, the guide assembly may comprise a housing that defines the plurality of guiding channels. Each of the guiding channels may extend from an entry hole on a top surface of the housing to an exit hole on a tissue contacting surface. The tissue contacting surface may be substantially opposite to the top surface. In yet still another exemplary aspect, at least one of the guiding channels may comprise an entry hole facing in a first axis on the top surface and an exit hole facing in a second axis on the tissue contacting surface. The first axis and the second axis may form an angle therebetween. The angle may range from about 15° to about 90 °.

According to an exemplary aspect, the tissue contacting surface may comprise multiple planes. In one aspect, the exit holes on the tissue contacting surface may be equally spaced. In another exemplary aspect, the entry holes on the top surface may be arranged in a first pattern and the exit holes on the tissue contacting surface may be arranged in a second pattern different from the first pattern. In still another exemplary aspect, the entry holes on the top surface may be arranged to receive the plurality of probes of the probe assembly. In yet still another aspect, the guide assembly may be custom made so that the tissue contacting surface of the housing closely matches the topography of a tissue surface of the patient to which the plurality of probes are to be placed.

According to some exemplary aspects, at least a portion of the tissue contacting surface may be curved. For example, at least a portion of the tissue contacting surface may comprise a geometric shape selected from the group consisting of: a convex shape; a concave shape; a wedge shape; and a flat shape.

In one exemplary aspect, at least one of the guiding channels may be curved. The curved guiding channel may be configured to guide a corresponding probe of the probe assembly along a predetermined tissue penetration trajectory.

In another exemplary aspect, the probe assembly and the guide assembly may be configured to engage one another. The probe assembly and the guide assembly may engage one another via at least one of: a snap-fastener; a screw; a magnet; and a glue or adhesive. In an exemplary embodiment, one of the probe assembly and the guide assembly may comprise a projecting member, and the other of the probe assembly and the guide assembly may comprise a corresponding hole to engage the projecting member. In another exemplary aspect, the engagement between the probe assembly and the guide assembly may be one of a permanent engagement and a detachable engagement. According to still another exemplary aspect, one of the probe assembly and the guide assembly may comprise a recess configured to receive the other of the probe assembly and the guide assembly.

In one aspect, the system may further comprise a second probe assembly. The guide assembly may be configured to guide the probes of the second probe assembly. In another exemplary aspect, the system may further comprise a second guide assembly configured to guide the plurality of probes of the probe assembly.

In still another exemplary aspect, the guide assembly may comprise a tubular housing. The tubular housing may be formed from a flexible, substantially flat body. The flat body may comprise a connecting member configured to connect ends of the flat body to form the tubular housing. In yet still another exemplary aspect, the tubular housing may be formed by two semi-circular portions connected together via a hinge.

According to some exemplary aspects, the tubular housing may define the plurality of guiding channels. At least one of the guiding channels may extend from an entry hole on an outer surface of the tubular housing to an exit hole on an inner surface of the tubular housing.

In another exemplary aspect, the system may further comprise a conduit for transmitting signals to an external device. The conduit may comprise at least one of a wire and a fiber optic. The conduit may be detachably connected to the probe assembly.

In still another exemplary aspect, the plurality of probes may be sized and configured to penetrate tissue of the patient's central or peripheral nervous system. In yet still another exemplary aspect, the plurality of probes may be sized and configured to penetrate tumor tissue or organ tissue.

Some exemplary aspects may provide a device for guiding a plurality of probes. The device may comprise a main body comprising a first surface having a plurality of first holes, a second surface having a plurality of second holes, and a plurality of guiding channels each extending between a respective first hole and a respective second hole. The guiding channels may be configured to guide a plurality of probes to desired tissue sites.

In another exemplary aspect, the second surface may comprise a tissue contacting surface. In still another exemplary aspect, the first holes on the first surface may be arranged in a first pattern, and the second holes on the second surface may be arranged in a second pattern, that is different from the first pattern.

According to one exemplary aspect, the first holes on the first surface may be arranged to receive the plurality of probes of a probe assembly. In another exemplary aspect, the main body may be configured to engage with a probe assembly containing the plurality of probes. The main body may be configured to engage with a plurality of probe assemblies each containing at least one probe. In one exemplary aspect, the main body may have a tubular shape. The first surface may be an outer surface of the main body, and the second surface may be an inner surface of the main body.

In an exemplary aspect, the main body may be formed from a flexible, substantially flat body. The flat body may comprise a connecting member configured to connect ends of the flat body to form the main body.

In another exemplary aspect, the main body may be formed by two semi-circular portions, each semi-circular portion having a first end and a second end, the first ends pivotally connected to each other. The main body may comprise a connecting member configured to connect the second ends of the two semi-circular portions together.

In still another exemplary aspect, the main body may further comprise an anchor for attaching the main body to tissue near the desired tissue sites. The anchor may comprise at least one tissue penetrating member. The anchor may comprise at least two tissue penetrating members.

In accordance with some exemplary aspects, an electrode array comprising a housing and a plurality of probes extending from the housing may be provided. At least one of the plurality of probes may be individually deployable from the housing. In an exemplary aspect, the at least one of the plurality of probes is retractable into the housing.

In another exemplary aspect, at least two of the plurality of probes may be simultaneously deployable from the housing. In still another exemplary aspect, at least one of the probes may be flexible or rigid. At least one of the probes has a resiliently biased shape. The resiliently biased shape may have a curved portion. In yet still another exemplary aspect, at least one of the probes may comprise a shape memory material. The shape memory material may comprise a shape memory alloy.

In one exemplary aspect, at least two of the probes may have lengths that are different from one another. Alternatively or additionally, at least two of the probes may have thicknesses that are different from one another.

In another exemplary aspect, at least one of the probes may comprise a first functional element and a second functional element. The first functional element may be different from the second functional element. At least one of the first and second functional elements may comprise an electrode. The electrode may be located at a distal tip of the probe.

According to still another exemplary aspect, the first functional element may be an electrode with a first set of characteristics, and the second functional element may be an electrode with a second set of characteristics. The characteristics may comprise at least one of: an impedance; a surface area; a material of construction; a surface texture; a porosity; a length; a width; a diameter; a thickness; a surface energy; a coating; and any combination thereof. At least one of the first and second functional elements may comprise at least one of a photodiode and a photosensor.

In one exemplary aspect, at least one of the probes may comprise a conductive trace. The conductive trace may be configured to mate with another trace disposed in the housing. In another exemplary aspect, at least one of the probes may comprise a hollow lumen along at least a portion of its length.

According to another exemplary aspect, the plurality of probes may be arranged in an array. In one exemplary aspect, the housing may comprise a probe deployment mechanism configured to move one or more of the plurality of probes relative to the housing. In another exemplary aspect, at least one of the probes is configured to be deployed after the housing is implanted on a tissue surface of a patient.

According to another aspect, the array may further comprise a guide assembly comprising a plurality of guiding channels configured to guide one or more of the plurality of probes to a desired tissue site. The guide assembly may comprise a tissue contacting surface, and at least one of the guiding channels may comprise a portion that is substantially parallel to at least a portion of the tissue contacting surface.

In another exemplary aspect, the guide assembly may comprise a tissue contacting surface, and at least one of the guiding channels may comprise a portion that forms an approximately 45° angle with respect to at least a portion of the tissue contacting surface.

In still another exemplary aspect, at least one of the guiding channels may be curved. The curved guiding channel is configured to guide a corresponding probe along a predetermined tissue penetration trajectory.

In yet still another exemplary aspect, at least one of the guiding channels or the corresponding probe received in the at least one of the guiding channels may comprise a trace configured to provide an electrical connection between the at least one of the guiding channels and the probe. Energy or signals may be transferred via the electrical connection.

According to another exemplary aspect, at least one of the guiding channels may comprise a first trace and the corresponding probe received in the at least one of the guiding channels may comprise a second trace. The first trace and the second trace may frictionally engage one another.

In still another exemplary aspect, the guide assembly may comprise a tissue contacting surface, and at least a portion of the tissue contacting surface may be curved. The portion of the tissue contacting surface may be custom made so that the tissue contacting surface closely matches the topography of a tissue surface to which the plurality of probes may be to be placed.

According to some exemplary aspects, the guide assembly may comprise a tissue contacting surface. At least a portion of the tissue contacting surface may comprise a geometric shape selected from the group consisting of: a convex shape; a concave shape; a wedge shape; and a flat shape.

In another exemplary aspect, the guide assembly may comprise a guide housing that defines the plurality of guiding channels, each of the guiding channels extending from an entry hole on a top surface of the guide housing to an exit hole on a tissue contacting surface. The exit holes on the tissue contacting surface may be arranged in array. The plurality of guiding channels may have at least 8 rows and at least 8 columns. In still another exemplary aspect, the exit holes on the tissue contacting surface may be equally spaced.

According to still yet another exemplary aspect, the guide assembly may comprise a tubular housing defining the plurality of guiding channels. At least one of the guiding channels may extend from an entry hole on an outer surface of the tubular housing to an exit hole on an inner surface of the tubular housing.

In one exemplary aspect, the array may further comprise a conduit for transmitting signals to an external device. The conduit may comprise at least one of a wire and a fiber optic. The conduit may be detachably connected to the housing.

In still another exemplary aspect, the housing may comprise at least one internal guiding lumen configured to receive one or more probes. The housing may comprise a drive assembly positioned adjacent the internal guiding lumen and may be configured to move one or more probes along the internal guiding lumen. The drive assembly may be manually controllable. Alternatively or additionally, the drive assembly may be remotely controllable or automatically controllable. The plurality of probes may comprise a signal detector, and at least one of the plurality of probes may be configured to move when a quality of a signal detected by the signal detector falls below a threshold level. In one exemplary aspect, the signal detected by the signal detector may comprise signals used in diagnosis of: obesity; an eating disorder; a neurological disorder; a stroke; a coma; amnesia; irregular blood flow in the brain; a psychiatric disorder; a cardiovascular disorder; an endocrine disorder; sexual dysfunction; incontinence; a hearing disorder; a visual disorder; a sleeping disorder; a movement disorder; impaired limb function; absence of a limb or a limb portion; a speech disorder; a physical injury; migraine headaches; stroke; a chronic or severe pain condition; or any combination thereof.

In another exemplary aspect, at least one of the plurality of probes may be configured to transmit a therapy signal, and at least one of the plurality of probes may be configured to move when a quality of the therapy signal falls below a threshold level. The therapy signal may comprise signals used in treatment of: obesity; an eating disorder; a neurological disorder; a stroke; a coma; amnesia; irregular blood flow in the brain; a psychiatric disorder; a cardiovascular disorder; an endocrine disorder; sexual dysfunction; incontinence; a hearing disorder; a visual disorder; a sleeping disorder; a movement disorder; impaired limb function; absence of a limb or a limb portion; a speech disorder; a physical injury; migraine headaches; stroke; a chronic or severe pain condition; or any combination thereof.

According to an exemplary aspect, the drive assembly may comprise a screw extending along at least a portion of the internal guiding lumen, a drive member configured to engage the one or more probes and the screw, and a drive mechanism configured to drive the drive member so as to move the one or more probes along the internal guiding lumen.

In another exemplary aspect, the drive assembly may comprise at least one pinch roller in contact with the one or more probes, and rotating the roller causes the one or more probes to move distally or proximally along the internal guiding lumen. The at least one pinch roller may be disposed adjacent the internal guiding lumen or may have a portion disposed in the internal guiding lumen. In still another exemplary aspect, the at least one pinch roller may comprise two pinch rollers.

In yet still another exemplary aspect, the drive assembly may comprise a gas discharging member having an outlet valve and being configured to discharge gas into the internal guiding lumen. Discharge of the gas may cause the one or more probes to advance the probe distally along the internal guiding lumen. The gas discharging member may comprise an electrolytic cell. The drive assembly may further comprise a gas suction member having an inlet valve and being configured to suction gas out of the internal guiding lumen, and suctioning of the gas may cause the one or more probes to retract proximally along the internal guiding lumen.

In some exemplary aspects, the drive assembly may comprise a suction member having an inlet valve and being configured to suction fluid out of the internal guiding lumen so as to retract the one or more probes proximally along the internal guiding lumen.

According to another exemplary aspect, the drive assembly may comprise an extendable piston having a distal end connected to the probe and a drive mechanism configured to extend or retract the extendable piston so as to move the probe distally or proximally along the internal guiding lumen. The drive mechanism may comprise at least one of a hydraulic drive element and a pneumatic drive element.

In still another exemplary aspect, the drive assembly may comprise a roller coupled to a proximal end of the one or more probes, a surface of the roller being in contact with an inner surface of the internal guiding lumen, and a controller configured to control rotation of the roller. In one aspect, rotation of the roller may cause the one or more probes to move distally or proximally along the internal guiding lumen. The drive assembly may further comprise a second roller coupled to the one or more probes.

In another exemplary aspect, the drive assembly may comprise a tube having inner threads and being disposed inside the internal guiding lumen, a screw attached to a proximal end of the one or more probes, the screw being configured to engage with and ride over the inner threads, and a drive mechanism configured to rotate at least one of the tube and the screw. Rotating at least one of the tube and the screw may cause the screw to move relative to the tube so as to move the one or more probes distally or proximally along the internal guiding lumen. In an exemplary embodiment, the drive mechanism may comprise a stepper motor.

In yet another exemplary aspect, the drive assembly may comprise first teeth disposed at least partially along the internal guiding lumen, a drive member coupled to the one or more probes, the drive member comprising a forward-moving member configured to engage or disengage at least one tooth of the first teeth when a first predetermined condition may be applied, such that when the forward-moving member engages and disengages the at least one tooth of the first teeth, the probe moves distally along the internal guiding lumen, and an actuator configured to actuate the forward-moving member so as to move the probe distally along the internal guiding lumen. The forward-moving member may comprise a shape memory material. In some aspects, the forward-moving member may be disengaged from the at least one tooth of the first teeth when the one or more probes moves proximally.

In another exemplary aspect, the drive assembly further may comprise second teeth disposed at least partially along the internal guiding lumen, and the drive member further may comprise a backward-moving member configured to engage or disengage at least one tooth of the second teeth when a second predetermined condition may be applied, so that when the backward-moving member engages and disengages the at least one tooth of the second teeth, the one or more probes moves proximally along the internal guiding lumen.

In one exemplary aspect, the drive assembly may comprise teeth disposed at least partially along the internal guiding lumen, a drive member coupled to the probe, the drive member comprising a backward-moving member configured to engage or disengage at least one tooth when a first predetermined condition may be applied, such that when the backward-moving member engages and disengages the at least one tooth, the one or more probes moves proximally along the internal guiding lumen, and an actuator configured to actuate the backward-moving member so as to move the one or more probes proximally along the internal guiding lumen. In one exemplary aspect, the backward-moving member may comprise a shape memory material. The backward-moving member may be disengaged from the at least one tooth when the one or more probes moves distally.

In another exemplary aspect, the drive assembly may comprise at least one first magnet disposed at least partially along the internal guiding lumen, a drive member coupled to the probe and comprising at least one second magnet, and a controller for energizing either the first magnet or the second magnet. Energizing either the first magnet or the second magnet may cause the one or more probes to move distally or proximally along the internal guiding lumen. In still another exemplary aspect, at least one of the first and second magnets may be configured to be activated by the controller. Alternatively or additionally, at least one of the first and second magnets may be an electromagnet or a permanent magnet. In some exemplary aspects, one of the first and second magnets may be an electromagnet, and the other of the first and second magnets may be a permanent magnet. In one aspect, at least one of the first and second magnets may comprise a plurality of magnets.

According to some exemplary aspects, the controller may be configured to energize either the first magnet or the second magnet by supplying electrical current to the magnet to be energized. The electrical current supplied in a first direction may cause the one or more probes to move distally along the internal guiding lumen, and the electrical current supplied in a second direction opposite the first direction may cause the one or more probes to move proximally along the internal guiding lumen.

In another exemplary aspect, the at least one first magnet or the at least one second magnet may comprise a plurality of magnets arranged in a row, and the plurality of magnets may be separated from one another by a predetermined distance. Energizing either the first magnet or the second magnet may cause the one or more probes to move a length that may be substantially equal to the predetermined distance.

According to one aspect, the housing may further comprise at least one of: a memory storage device; a signal processing unit; a power transfer device; a power conversion device; a wireless communication device; a CPU; a microcontroller; a drug delivery assembly or reservoir; an electromagnetic field generator; a light source; a camera assembly; an impedance measurement device; a radiopaque marker; and a power supply.

In one exemplary aspect, the housing may further comprise a power transfer device configured to convert non-electrical energy to electrical energy. In another aspect, the housing may comprise a wireless communication device configured to transfer information via: radiofrequency; microwave; infrared; ultrasound; or any combination thereof. In still another aspect, the housing may comprise a signal processing element configured to perform a signal processing function selected from the group consisting of: amplification; filtering; sorting; conditioning; translating; interpreting; encoding; decoding; combining; extracting; sampling; multiplexing; analog to digital converting; digital to analog converting; mathematically transforming; and any combination thereof.

In some exemplary aspects, at least one of the plurality of probes may comprise one or more reservoirs or ports for delivery of an agent. The agent may be a fluid. In another exemplary aspect, the array may further comprise a pump configured to supply the agent to the one or more reservoirs or ports. At least one of the one or more reservoirs or ports may be refillable.

In another exemplary aspect, the housing and the plurality of probes may be a micro electromechanical system. The micro electromechanical system may be integrated into a silicon substrate. In still another exemplary aspect, at least one of the plurality of probes may comprise at least one electrode.

According one aspect, at least one of the plurality of probes may comprise at least one of: a recording electrode; a stimulating electrode; a sensor; an acoustic transducer; a light source; a heat source; a cooling source; an agent eluding port; and a reservoir.

In another exemplary aspect, the array may further comprise an anchor for anchoring the array to a tissue surface to which the plurality of probes may be to be inserted. The anchor may comprise at least one tissue penetrating member. In some exemplary embodiments, the anchor may comprise at least two tissue penetrating members.

Various exemplary aspects of the invention may provide a kit used for implanting an electrode system. The kit may comprise: a probe assembly comprising a plurality of probes configured to penetrate tissue of a patient; and a first guide assembly and a second guide assembly. Each of the first and second guide assemblies may comprise a housing defining a plurality of guiding channels, and each of the guiding channels may extend between an entry hole on a first surface of the housing and an exit hole on a second surface of the housing. The entry holes of the first guide assembly and the entry holes of the second guide assembly may be arranged in substantially identical patterns. The second surface of the first guide assembly has a characteristic differing from that of the second surface of the second guide assembly.

According to another exemplary aspect, each of the first surfaces of the first and second guide assemblies may be configured to engage with the probe assembly. In still another exemplary aspect, the entry holes on each of the first surfaces of the first and second guide assemblies may be arranged such that, when the probe assembly engages with one of the first and second guide assemblies, the plurality of probes may be inserted into the entry holes.

In some exemplary aspects, the characteristic may be a contour of the second surface of the first guide assembly that is different from a contour of the second surface of the second guide assembly. Alternatively or additionally, the characteristic may be an arrangement of the exit holes on the second surface of the first guide assembly that may be different from an arrangement of the exit holes on the second surface of the second guide assembly.

According to another exemplary aspect, the first and second guide assemblies may be configured such that each of the second surfaces may be contoured to substantially match a different tissue surface of a patient. According to still another aspect, the first and second guide assemblies may be custom made so that second surfaces may be contoured to substantially match a different tissue surface of a particular patient.

In some exemplary aspects, at least one of the plurality of guiding channels in at least one of the first and second guide assemblies may be curved. According to another exemplary aspect, at least one of the first and second guide assemblies may comprise a recess configured to receive the probe assembly. In one exemplary aspect, the plurality of guiding channels in at least one of the first and second guide assemblies may be configured to guide the probes in different penetration trajectories.

In another exemplary aspect, the probe assembly and the guide assembly may be configured to engage one another. According to some exemplary aspects, the probe assembly and the guide assembly may engage one another via at least one of: a snap-fastener; a screw; a magnet; and a glue or adhesive. One of the probe assembly and the guide assembly may comprise a projecting member, and the other of the probe assembly and the guide assembly may comprise a corresponding hole to engage the projecting member.

In another exemplary aspect, the engagement between the probe assembly and the guide assembly may be one of a permanent engagement and a detachable engagement.

According to still another exemplary aspect, the kit may further comprise a signal processing element configured to perform a signal processing function selected from the group consisting of: amplification; filtering; sorting; conditioning; translating; interpreting; encoding; decoding; combining; extracting; sampling; multiplexing; analog to digital converting; digital to analog converting; mathematically transforming; and any combination thereof. In one exemplary aspect, the kit may further comprise a communication device configured to send and/or receive signals from and/or to the signal processing element.

In some aspects, the kit may further comprise at least one of a therapeutic device or a diagnostic device configured to communicate with the communication device.

Another exemplary aspect may provide a method of inserting a probe assembly into a patient. The method may comprise providing any of the exemplary kits described above, determining a topography of a tissue surface into which the probe assembly is to be inserted, selecting at least one of the first and second guide assemblies that closely matches the topography of the tissue surface, placing the selected guide assembly onto the tissue surface with the second surface in contact with the tissue surface, and inserting the plurality of probes into the entry holes on the first surface.

In one exemplary aspect, at least one of the guide assemblies may be custom made to match the topography. In another aspect, determining the topography may comprise performing at least one of: a magnetic resonance imaging (MRI), a functional MRI, a computed tomography (CT-scan), an ultrasound imaging procedure, an X-ray imaging, or a fluoroscopy.

According to still another exemplary aspect, a method of implanting a plurality of probes into a patient may be provided. The method may comprise: providing a plurality of probes; determining a topography of a tissue surface into which the probes are to be inserted; providing a guide assembly comprising a first surface having a plurality of entry holes configured to receive the plurality of probes, a second surface having a plurality of exit holes, the second surface having a surface contour substantially matching the topography of the tissue surface, and a plurality of guiding channels each extending from a corresponding entry hole on the first surface to a corresponding exit hole on the second surface; bringing the second surface of the guide assembly in contact with the tissue surface; and inserting the probes into the entry holes of the guide assembly.

According to another aspect, the plurality of probes may be arranged in one or more probe assemblies. One of the probe assembly and the guide assembly may comprise a recess configured to receive the other of the probe assembly and the guide assembly.

In some exemplary aspects, the guide assembly may be custom made so that at least the second surface of the guide assembly substantially matches the topography of the tissue surface.

In one exemplary aspect, determining the topography may comprise performing at least one of: a magnetic resonance imaging (MRI), a functional MRI, a computed tomography (CT-scan), an ultrasound imaging procedure, an X-ray imaging, or a fluoroscopy.

In another exemplary aspect, at least one of the plurality of guiding channels may be curved. In still another exemplary aspect, the plurality of guiding channels are configured to guide the probes in different penetration trajectories.

In one exemplary aspect, the plurality of probes may be arranged in a housing, and at least one of the plurality of probes may be individually deployable from the housing. The housing may comprise a probe deployment mechanism configured to move the at least one of the plurality of probes relative to the housing.

In another exemplary aspect, the entry holes on the first surface may be arranged in a first pattern, and the exit holes on the second surface may be arranged in a second pattern that may be different from the first pattern.

In various exemplary aspects, at least one of the probes may be movable relative to another of the probes.

In another exemplary aspect, the method may further comprises moving at least one of the probes relative to another of the probes after inserting the probes into the entry holes of the guide assembly.

One exemplary aspect of the invention may provide a method of implanting an electrode sensor system. The method may comprise: providing an electrode system comprising at least one probe, a processing unit, and a conduit for transmitting signals between the probe and the processing unit; creating an opening in the skull; inserting the probe through the opening; placing the processing unit on an external portion of the skull; creating a slot on the surface of the skull, the slot extending at least partially from the opening to the processing unit; and placing the conduit in the slot.

In another exemplary aspect, inserting the probe through the opening may comprise inserting the probe at least partially into the brain. In still another aspect, the at least one probe may comprise a plurality of probes. At least one of the plurality of probes may comprise at least one of: a recording electrode; a stimulating electrode; a sensor; an acoustic transducer; a light source; a heat source; a cooling source; an agent eluding port; and a reservoir. At least one of the plurality of probes may comprise at least one electrode. In yet still another exemplary aspect, the method may further comprise connecting the conduit to one or more additional probes.

In some exemplary aspects, the probe may be configured to record cellular activity. Alternatively or additionally, the probe may be configured to deliver energy to tissue. The energy delivered may comprise at least one selected from the group consisting of: heat energy; cryogenic energy; light energy; radiation energy; chemical energy; mechanical energy; electrical energy; and any combination thereof.

In another exemplary aspects, the probe may be configured to deliver agent. The agent may comprise a pharmaceutical agent.

According to still another exemplary aspect, the probe may comprise a sensor. The sensor may comprise at least one selected from the group consisting of: a thermal sensor; a pressure sensor; a chemical sensor; a force sensor; an electromagnetic field sensor; a physiologic sensor; a photodetector; a pH sensor; an oxygen sensor; a blood sensor; an electrode; and any combination thereof.

In another exemplary aspect, the processing unit may be located less than 20 cm from the sensor. In still another exemplary aspect, the method may further comprise placing the processing unit on the top of the skin of the patient. According to another aspect, the method may comprise placing the processing unit on top of the skull of the patient under the scalp.

According to still another exemplary aspect, a method of implanting a plurality of probes into a patient may be provided. The method may comprise: providing a probe assembly having a main body and a plurality of probes extending from the main body, at least one of the plurality of probes being movable relative to the main body; inserting the plurality of probes into tissue of the patient; detecting signals with the at least one of the plurality of probes; and selectively moving the at least one of the plurality of probes relative to the main body until the at least one of the plurality of probes detects signals having a desired signal strength.

In some exemplary aspects, selectively moving may be controlled automatically. Alternatively or additionally, selectively moving may be controlled manually and/or remotely. According to another aspect, selectively moving may comprise advancing or retracting the at least one of the plurality of probes.

In still another exemplary aspect, the method may further comprise transmitting stimulating signals into the tissue. Detecting signals may comprise detecting signals from the tissue responsive to the stimulating signals.

According to one exemplary aspect, selectively moving the at least one of the plurality of probes may be performed after the step of inserting the plurality of probes into tissue of the patient.

In another exemplary aspect, the desired signal strength may be above a predetermined threshold level. In another exemplary aspect, the method may further comprise adjusting the predetermined threshold level.

According to another exemplary aspect, the method of implanting a plurality of probes into a patient may comprise: providing a probe assembly having a main body and a plurality of probes extending from the main body, at least one of the plurality of probes being movable relative to the main body; inserting the plurality of probes into tissue of the patient; transmitting therapeutic signals to the tissue with the at least one of the plurality of probes; and selectively moving the at least one of the plurality of probes relative to the main body until a desired therapeutic result is achieved.

In another exemplary aspect, the desired therapeutic result may comprise prevention or reduction of a seizure or improvement in motor function of a patient in response to the therapeutic signals.

In still another exemplary aspect, the method may further comprise observing the patient's condition relating to the desired therapeutic result. The observing may be performed with at least one sensor selected from the group consisting of: a thermal sensor; a pressure sensor; a chemical sensor; a force sensor; an electromagnetic field sensor; a physiologic sensor; a photodetector; a pH sensor; an oxygen sensor; a blood sensor; and any combination thereof.

In yet still another exemplary aspect, the method may further comprise stopping the selective movement of the at least one of the plurality of probes when a change in the patient's condition exceeds a predetermined threshold level. The observing may be performed by a visual observation of the patient.

In accordance with some exemplary embodiments, the desired therapeutic result may be above a predetermined threshold level. The method may further comprise adjusting the predetermined threshold level.

One exemplary aspect of the invention may provide a system comprising any of the exemplary electrode system described above and a functional device associated with the electrode system. Another exemplary aspect of the invention may provide a system comprising any of the electrode array described above and a functional device associated with the electrode array.

In still another exemplary aspect, at least one of the plurality of probes may comprise a sensor configured to detect signals generated from one or more living cells. The functional device may be controllable by a control signal generated based on the signals detected by the sensor.

According to another exemplary embodiment, the system may further comprise a processing unit configured to receive the detected signals to produce processed signals. The processing unit may receive the detected signals wirelessly. The processed signals may comprise the control signal. The processing unit may be configured to transmit the control signal to the functional device wirelessly.

In another exemplary aspect, the processing unit may be implanted in the patient's body. In still another exemplary aspect, the processing unit may be placed external to the patent's body. In yet still another exemplary aspect, the processing unit may be configured to perform at least one of: amplification; filtering; sorting; conditioning; translating; interpreting; encoding; decoding; combining; extracting; sampling; multiplexing; analog to digital converting; digital to analog converting; and mathematically transforming. In yet still another exemplary aspect, the functional device may be controlled by control signals generated under voluntary control of the patient.

In some exemplary aspects, the functional device may be configured to receive wireless signals from the probe system. At least one of the plurality of probes may be configured to send signals to one or more living cells. The functional device may be configured to transmit the signals to the at least one of the plurality of probes.

According another exemplary aspect, the system may further comprise a processing unit configured to transmit the signals to the at least one of the plurality of probes. The processing unit may be configured to perform at least one of: amplification; filtering; sorting; conditioning; translating; interpreting; encoding; decoding; combining; extracting; sampling; multiplexing; analog to digital converting; digital to analog converting; and mathematically transforming.

In still another exemplary aspect, the signals may be configured to polarize, stimulate, or affect the one or more living cells. For example, the signals may comprise at least one of: electric current, an electromagnetic field, acoustic energy, heat energy, cooling energy, pharmaceutical drug or agent, light, and mechanical vibration.

In accordance with some exemplary embodiments, the functional device may comprise at least one of: a therapeutic device; a restorative device; and diagnostic device. The therapeutic device may be configured to perform a therapeutic function comprising a treatment of one or more of: obesity, an eating disorder, a neurological disorder, a psychiatric disorder, a cardiovascular disorder, an endocrine disorder, sexual dysfunction, incontinence, a hearing disorder, a visual disorder, a sleeping disorder, a movement disorder, a speech disorder, physical injury, migraine headaches, stroke, and chronic pain.

In one exemplary aspect, the diagnostic device may be configured to perform a patient diagnosis comprising a diagnosis of one or more of: obesity, an eating disorder, a neurological disorder, a psychiatric disorder, a cardiovascular disorder, an endocrine disorder, sexual dysfunction, incontinence, a hearing disorder, a visual disorder, sleeping disorder, a movement disorder, a speech disorder, physical injury, migraine headaches, stroke, and chronic pain.

In still another exemplary aspect, the restorative device may be configured to restore a bodily function of the patient, the bodily function comprising one or more of vision, hearing, speech, communication, limb motion, ambulation, reaching, grasping, standing, rolling over, bowel movement, and bladder evacuation.

According yet still another exemplary aspect, the functional device may be implanted in the patient's body or placed external to the patent's body.

In one exemplary aspect, the functional device may be controlled by control signals generated under voluntary control of the patient. In still another exemplary aspect, the functional device may comprise at least one selected from the group consisting of: a computer, a computer display, a mouse, a cursor, a joystick, a personal data assistant, a robot or robotic component, a computer controlled device, a teleoperated device, a communication device, a vehicle, an adjustable bed, an adjustable chair, a remote controlled device, a Functional Electrical Stimulator device, a muscle stimulator, an exoskeletal robot brace, an artificial or prosthetic limb, a vision enhancing device, a vision restoring device, a hearing enhancing device, a hearing restoring device, a movement assist device, a medical therapeutic equipment, a drug delivery apparatus, a medical diagnostic equipment, a bladder control device, a bowel control device, a human enhancement device, and a closed loop medical equipment.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate various embodiments consistent with the invention, and, together with the description, serve to explain the principles of the invention.

FIG. 1 is a schematic illustration of an electrode array system, according to an exemplary embodiment of the invention, showing a probe assembly and a guide assembly separately.

FIG. 2 is a schematic illustration of the electrode array system of FIG. 1, showing the probe assembly and the guide assembly engaged to each other.

FIG. 3 is a schematic illustration of an electrode array system, according to another exemplary embodiment of the invention.

FIG. 4 is a schematic illustration of an electrode array system, according to still another exemplary embodiment of the invention.

FIG. 5 is a schematic illustration of a guide assembly, according to another exemplary embodiment of the invention.

FIG. 6 is a schematic illustration of the guide assembly shown in FIG. 5 with corresponding probe assemblies engaged or being engaged therewith, according to another exemplary embodiment of the invention.

FIG. 7 is a schematic illustration of an electrode array system, according to another exemplary embodiment of the invention.

FIG. 8 is a schematic illustration of a guide assembly shown in FIG. 7, prior to being formed into a tubular configuration, according an exemplary embodiment of the invention.

FIG. 8A is a schematic illustration of a guide assembly shown in FIG. 7, prior to being formed into a tubular configuration, according another exemplary embodiment of the invention

FIG. 9 is a cross-section view along the IX-IX plane of FIG. 7.

FIG. 10 is a schematic illustration of a probe assembly, according to another exemplary embodiment of the invention.

FIG. 11 is a plan view of the probe assembly shown in FIG. 10 in the direction from the bottom.

FIG. 12-19, 19A, 19B, and 20 are schematic illustrations of linear drive assemblies, according to various exemplary embodiments of the invention.

FIG. 21 is a schematic illustration of an electrode array system implanted on a patient's brain, according to an exemplary embodiment of the invention.

FIG. 22 is a schematic illustration of a brain implant apparatus, according to an exemplary embodiment of the invention.

FIG. 23 is a schematic illustration of a brain implant apparatus, being applied to a patient.

FIG. 24 is a cross-section view of the brain implant apparatus shown in FIG. 23, illustrating the positioning of various elements of the brain implant apparatus, according to an exemplary embodiment of the invention.

### DESCRIPTION OF THE EMBODIMENTS

Reference will now be made in detail to the exemplary embodiments consistent with the present invention, examples of which are illustrated in the accompanying drawings. Wherever possible, the same reference numbers will be used throughout the drawings to refer to the same or like parts.

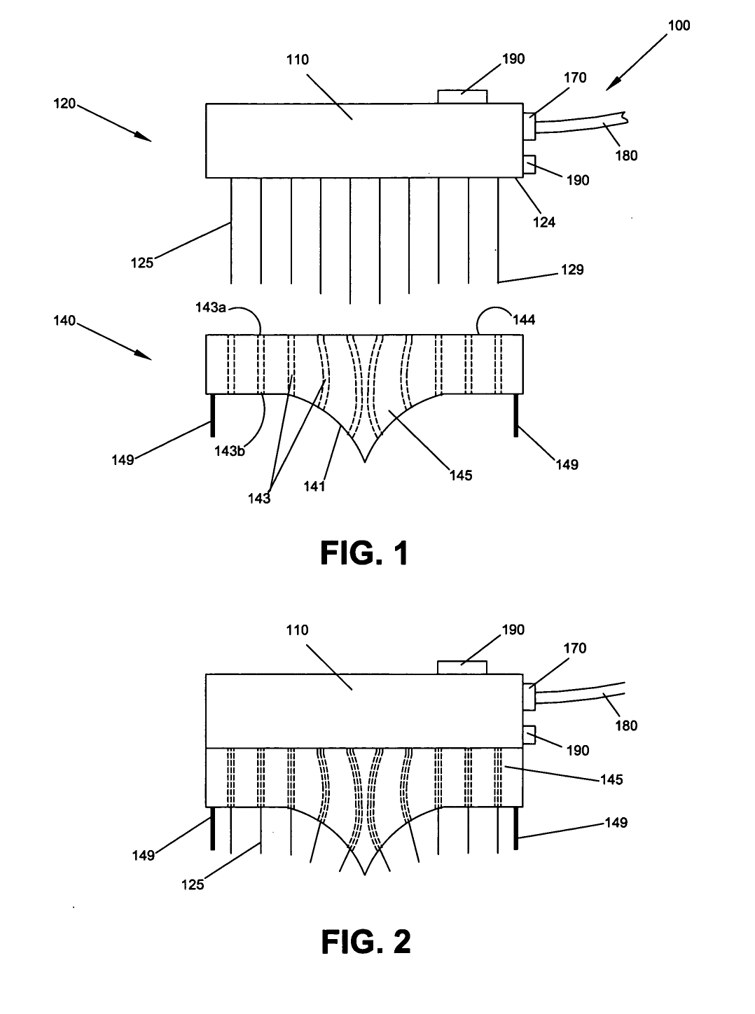

FIGS. 1 and 2 show an electrode array system 100 to be implanted within a patient's body, such as, for example, in the central or peripheral nervous system, according to an exemplary embodiment of the invention. The system 100 may comprise a probe assembly 120 having a plurality of probes 125 and a guide assembly 140 configured to guide the plurality of probes 125 into a desired tissue site (e.g., neural tissue of the brain).

The plurality of probes 125 may be configured to detect electrical signals or impulses (e.g., electrical neural signals generated from neurons or other living cells) from the patient's body and may be arranged in an array, for example, in a 10×10, 8×8, or 5×5 matrix. The array may have a square, rectangular, or circular pattern, or any other desired pattern. The probes 125 may have similar or dissimilar geometries, such as, lengths and diameters. Alternatively or additionally, the probes 125 may have similar or dissimilar properties, such as level of rigidity.

Each probe 125 may have an active electrode 129, preferably at its distal end portion, that may be electrically isolated from neighboring electrodes 129 by a suitable non-conducting material. For example, the probe 125 may be a wire insulated up to its distal end portion. In some exemplary embodiments, at least one of the probes 125 may have multiple electrodes 129 along its length. The multiple electrodes may have similar or dissimilar geometries (e.g., surface area, surface contour, length, width, diameter, thickness, etc.) or similar or dissimilar material properties (e.g., impedance, material of construction, surface texture, porosity, coating, surface energy, etc.).

As shown in FIGS. 1 and 2, the probe assembly 120 may comprise a signal transfer conduit 180, such as, for examples, a bundle of wires or optical fibers, for transmitting and/or receiving signals to and/or from an external device (e.g., a processing unit for receiving, storing, and/or processing signals and/or transmitting signals to the electrodes and various medical devices in the operating room), another implanted device, and/or another probe assembly. The conduit 180 may be detachably connected to the probe assembly 120 via a suitable detachable connector 170. Alternatively, the conduit 180 may be permanently fixed to the probe assembly 120. In this embodiment, a free end of the conduit 180 may have a suitable connector for connecting to at least one of the above-mentioned devices.

In an exemplary embodiments, the probe assembly 120 may comprise one or more connectors 190 for connecting to multiple devices. In some exemplary embodiments, each of the electrodes 129 may be connected to an individual wire that may be bundled together in the conduit 180, so that the electrodes 129 may individually transmit and/or receive signals. Alternatively or additionally, the probe assembly 120 may comprise a wireless transfer device (not shown) for transmitting and/or receiving signals to and/or from an external device, another implanted device, and/or another probe assembly. The wireless transfer device may utilize one or more of, for example, radiofrequency, infrared, ultrasound, or microwave communication module or any other wireless communication module known in the art. Alternatively or additionally, the probe assembly 120 may comprise a signal processing unit including a signal multiplexing function such that the conduit 180 may include a number of conductors less than the total number of the electrodes 129. Electrical to optical conversion may be included such that the conduit 180 may include an optical fiber for signal and/or power transmissions.

The electrodes 129 may be configured to detect cellular signals (e.g., multicellular signals), and the probes 125 may include wires or traces attached to the electrodes 129 to send detected signals and/or receive signals from one or more external devices. The term “cellular signals,” as used herein, refers to signals or combination of signals that may emanate from any living cell, such as, for example, subcellular signals, intracellular signals, and extracellular signals.

For example, “cellular signals” may include, but not be limited to: neural signals (e.g., neuron action potentials or spikes, local field potential (LFP) signals, electroencephalogram (EEG) signals, electrocorticogram signals (ECoG), and signals whose frequency range falls between single neuron spikes and EEG signals); cardiac signals (e.g., cardiac action potentials); electromyogram (EMG) signals; glial cell signals; stomach cell signals; kidney cell signals; liver cell signals; pancreas cell signals; osteocyte cell signals; sensory organ cell signals (e.g., signals emanating from the eye or inner ear); tumor cell signals; and tooth cell signals.

The term “multicellular signals,” as used herein, refers to signals emanating from two or more cells, or multiple signals emanating from a single cell. The term “subcellular signals,” as used herein, refers to, for example, a signal derived from a part of a cell, a signal derived from one particular physical location along or within a cell, a signal from a cell extension (e.g., dendrite, dendrite branch, dendrite tree, axon, axon tree, axon branch, pseudopod, or growth cone), and signals from organelles (e.g., golgi apparatus or endoplasmic reticulum). The term “intracellular signals,” as used herein, refers to a signal that is generated within a cell or by the entire cell that is confined to the inside of the cell up to and including the membrane. The term “extracellular signals,” as used herein, refers to signals generated by one or more cells that occur outside of the cell(s).

The probes 125 may have a variety of different types of electrodes or other functional elements, such as, for example, recording electrodes, stimulating electrodes, sensors (e.g., a photo sensor, a pressure sensor, a force sensor, an electromagnetic field sensor, a physiologic sensor such as an EKG sensor or a blood glucose sensor, a photo sensor, a pH sensor, an oxygen sensor, a blood sensor, etc.), transducers (e.g., acoustic transducers), or any combination thereof. The differences between these different types of electrodes or functional elements may include different materials of construction, coatings, thicknesses, lengths, diameters, geometric shapes, etc. In some exemplary embodiments, each of the recording electrodes 129 may form a recording channel that may directly detect electrical signals generated from each of the neurons in the electrode's vicinity.

In one exemplary embodiment, one or more probes 125 may comprise a photodiode for transmitting light (e.g., ultraviolet light) for stimulation of cells. In another exemplary embodiment, one or more probes 125 may comprise a hollow space (e.g., a fluid reservoir) for storage and/or delivery of therapeutic agents or drugs. For example, an exemplary array disclosed in copending U.S. patent application Ser. No. 10/717,924 by Donoghue et al., the entire disclosure of which is incorporated by reference herein, may be used in connection with various systems and methods of this invention. In still another exemplary embodiment, one or more probes 125 may include a photodiode-transistor pair for transmitting light and detecting reflective light indicative of cellular signals.

As discussed above, the probes 125 may transmit the detected signals to another device via one or more conduits 180, such as wires or a wireless transmission module. For example, the probes 125 may transmit the detected signals to a processing unit, and the processing unit may preprocess the received signals (e.g., impedance matching, noise filtering, and/or amplifying), digitize them, and further process the signals to extract neural information that it may transmit to an external computing device. Alternatively or additionally, the processing unit may transmit signals, energy, and/or one or more therapeutic agents or drugs to one or more probes 125 so as to, for example, polarize or stimulate the neighboring nerves or cells or activate the delivery of the therapeutic agents or drugs, if applicable. In an exemplary embodiment, the processing unit may transmit energy (e.g., passing electric current or applying an electromagnetic field) to a probe 125 to polarize, stimulate, or otherwise affect the nerves around the probe 125. In some exemplary embodiments, the transmitted energy may improve or otherwise modify recorded information received simultaneously with or subsequent to the energy transmission.

The probe assembly 120 may be used as a part of a therapeutic and/or diagnostic system. For example, the probe assembly 120 may be used to treat or diagnose one or more of the following: obesity; an eating disorder; a neurological disorder such as epilepsy or Parkinson's Disease; a stroke; a coma; amnesia; irregular blood flow in the brain; a psychiatric disorder such as depression; a cardiovascular disorder; an endocrine disorder; sexual dysfunction; incontinence; a hearing disorder; a visual disorder; a sleeping disorder; a movement disorder; impaired limb function; absence of a limb or a limb portion; a speech disorder such as stuttering; a physical injury; a migraine headache; stroke; or chronic or temporary pain.

Alternatively or additionally, the probe assembly 120 may be used as a part of a system which may restore patient function, including, but not limited to one of more of the following: vision; hearing; speech; communication; limb motion; ambulation; reaching; grasping; standing; sitting; rolling over; bowel movement; and bladder evacuation.

Referring to FIGS. 1 and 2, the probe assembly 120 may comprise a housing 110 from which the plurality of probes 125 may project to contact or penetrate into tissue of a patient. The probes 125 may be placed such that electrodes or other functional elements carried by the probes 125 may be positioned in proximity to one or more desired living cells. For example, the probes 125 may be inserted into numerous types of tissue, including but not limited to: brain tissue; other nerve tissue (e.g., peripheral nerve tissue or nerves of the spine); organ tissue (e.g., heart, pancreas, liver, or kidney tissue); tumor tissue (e.g., brain tumor or breast tumor tissue); prostate tissue; and any combination thereof.

As will be described further herein, the probes 125 may be individually deployable (e.g., advanced and/or retracted relative to the bottom of the housing 110) from the housing 110 during and/or after surgery.

The probes 125 may be rigid or flexible. For example, the probes 125 may be flexible, yet sufficiently rigid so as to penetrate tissue. The tissue may include: central nervous tissue such as spinal tissue and brain tissue; peripheral nerve tissue; tumor tissue such as brain or breast tumor tissue; and organ tissue such as heart, kidney, pancreas, prostate, or liver tissue. In some exemplary embodiments, the probes 125 may have a resiliently biased shape, such as, for example, a straight or curved shape. The probes 125 may be made of a variety of materials. For example, the probes 125 may be made of silicon, metal, or plastic material, or any combination thereof. In an exemplary embodiment, the probes 125 may be made of shape memory material, such as, for example, Nitinol or a shape memory polymer. Any other resilient metal or alloy may be used additionally or alternatively.

In some exemplary embodiments, some of the probes 125 may be made of relatively rigid materials, and some of the probes 125 may be made of relatively flexible materials. In another exemplary embodiment, at least one of the probes 125 may be made of materials sufficiently rigid and configured in specific construction geometries such that the resultant column strength of a probe 125 may support penetration through the particular types of tissue into which the probe 125 is to be inserted.

Alternatively or additionally, some of the probes 125 may be made of combinations of a relatively rigid material and a relatively flexible material. For example, a single probe 125 may have, along its length, at least one portion made of a relatively rigid material and at least one portion made of a relatively flexible material. The portion made of a relatively rigid material may provide sufficient column strength to enable insertion of the probe 125 into tissue. The portion made of a relatively flexible portion may allow passing of the probe 125 through a curved guiding channel of the guide assembly 140 or bending of the probe 125 in tissue. Alternative, or in addition, to the portion made of a relatively flexible portion, the probes 125 may have one or more joints or flexing/bending portions to facilitate the bending.

The lengths of the probes 125 may vary within a probe assembly 120. Alternatively, the probes 125 within a probe assembly 120 may have substantially the same lengths. In general, the length of the probes 125 may vary depending on the type or location of tissue into which the probes 125 are intended to be inserted. By way of example only, the lengths of the probes 125 (e.g., the length of the probe 125 extending beyond the housing 145 when the probe 125 is fully inserted) may range from about 0.3 mm to about 2.5 mm. Preferably, the lengths may range from about 0.5 mm to about 1.5 mm. In some exemplary embodiments, the probes 125 may have a length greater than 2.5 mm (e.g., 5˜50 cm for various applications). By way of examples only, the probes 125 may have a length of about 10 cm for deep brain, about 20 cm for organs, and about 50 cm for tumors.

The probe assembly 120 having relatively longer probes 125 may have a variety of applications. For example, the probe assembly 120 may be placed on a surface of the brain to allow one or more probes 125 to extend deep into the brain for a procedure known as deep brain stimulation, for example. The probe assembly 120 may also be placed on an organ (e.g., heart, pancreas, kidney, liver, etc.) or a tumor (e.g., brain tumor or breast tumor), where the probes 125 may penetrate deep into the desired tissue of the organ or tumor.

The guide assembly 140 may include a housing 145 that may define a plurality of guiding channels 143 (e.g., trajectory lumens defining the tissue penetration trajectories) configured to guide the plurality of probes 125 to desired tissue sites. Each of the guiding channels 143 may extend from an entry hole 143a on a top surface 144 of the housing 145 to an exit hole 143b on a tissue contacting surface 141, and may include traces to send and/or receive signals from frictional engagement with the probes 125 guided therein. Alternatively or additionally, at least one of the guiding channels 143 or the corresponding one or more probes 125 received in the guiding channel 143 may include a conductive trace to provide an electrical connection between the at least one of the guiding channels 143 and the one or more probes 125. In some exemplary embodiments, energy and/or signals may be transferred via the electrical connection provided by the conductive trace. In an exemplary embodiment, a single guiding channel 143 may be configured to allow two or more probes 125 to advance and/or retract therealong.

The exit holes 143b may be equally spaced from one another or may be spaced in any desired pattern. The trajectory of a probe 125 into tissue may be determined primarily by trajectory of the guiding channel through which the probe 125 may be guided and the rigidity and biased shape of the probe 125.

In some exemplary embodiments, at least a portion of the housing 145 may have a coating to promote or prevent tissue ingrowth or a coating to enhance biocompatibility. Alternatively or additionally, at least a portion of the housing 145 may be made of a porous material such as to promote tissue ingrowth. Any other coating material known in the implant art may also be used alternatively or additionally.

As will be described further herein, the housing 145 may have various geometries, especially in the tissue contacting surface 141 which accommodates tissue contours. The housing 145 may have variable heights (i.e., distance between the top surface 144 and the tissue contacting surface 141) that may determine how far the probes 125 penetrate into tissue. In an exemplary embodiment, the array system 100 may have a plurality of housings 145 with the identical entry hole pattern and exit hole patterns, but with different heights so as to have different penetration depths of the probes 125 in the same pattern.

According to various exemplary embodiments, the guide assembly 140 may be made of a relatively rigid or semi-rigid biocompatible material (e.g., sufficiently rigid so that the material does not deform when guiding the plurality of probes 125). For example, the guide assembly 140 may be made of, at least partially, a plastic material (e.g., delrin or polysulfone), silicon or silicon-based composites, glass, or graphite composites. In another exemplary embodiment, the guide assembly 140 may be constructed of a plurality of separate pieces.

According to some exemplary embodiments, the guide assembly 140 may be made of a material used in stereolithography or other rapid manufacturing process. As will be described further herein, when the guide assembly 140 is custom made to closely match the topography of the patient's tissue surface, this type of material may facilitate such a custom-making process of the guide assembly 140.

The plurality of guiding channels 143 may be coated and/or lubricated with a suitable material (e.g., Teflon) to reduce friction. In some exemplary embodiments, the guiding channels 143 may be treated with a relatively hard (e.g., durable) material to substantially prevent damage to the channels 143 by the probes 125.

As shown in FIG. 1, the top surface 144 of the housing 145 may be configured to mate with the bottom surface 124 of the housing 110 of the probe assembly 120, and the pattern of the entry holes 143a on the top surface 144 may match the pattern of the probes 125 of the probe assembly 120 so that each guiding channel 143 may individually guide each corresponding probe 125 to a particular, desired tissue site.

The tissue contacting surface 141 of the guide assembly 140 may have a variety of different shapes and sizes depending on the shape of the tissue surface to which the probes 125 are to be inserted. For example, the guide assembly 140 may have a flat, convex, concave, or wedge-shaped surface, or any other suitable shaped surface. In some exemplary embodiments, as will be described further herein, the guide assembly 140 may have a tubular shape configured such that it may be implanted to surround a peripheral nerve or other tubular shaped tissue portion.

The guide assembly 140 may also be custom made to closely match the topography of the tissue surface (e.g., sulcus of the brain). In such embodiments, the topography of the tissue surface first may be determined by performing any suitable, known visualization method, such as, for example, a magnetic resonance imaging (MRI), a functional MRI, a computed tomography (CT-scan), an ultrasound imaging procedure, X-ray, and fluoroscopy. Once the topography of the tissue surface is determined, the tissue contacting surface 141 may be machined, molded, constructed with a layer additive process (e.g., stereolithography), or otherwise manufactured in accordance with the determined topography of the tissue surface. While the electrode array system 100 may have different types of guide assemblies 140 depending on the shape of the tissue surface, the same probe assembly 120 may be used for different types of guide assemblies 140.

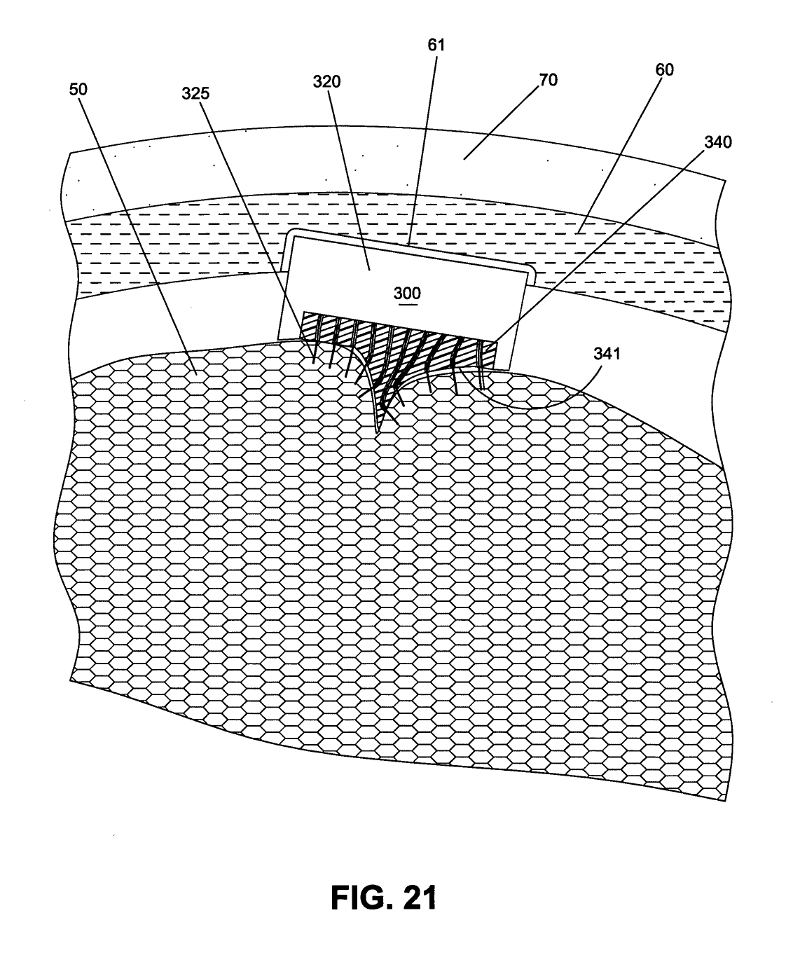

As shown in FIGS. 1 and 2, at least some of the guiding channels 143 may be curved so that, as will be described later with reference to FIG. 21, the guided probes 125 may penetrate into tissue sites that may otherwise be inaccessible to straight probes. For example, a desired tissue site into which a probe 125 is to be placed may be located on a side surface of a crevice or sulcus of a patient's body (e.g., brain), which may extend substantially along the longitudinal direction of the probe insertion. In that case, if the probe 125 is substantially straight, the probe 125 may not be accurately positioned in the desired tissue site. The curved guiding channel 143 thus may facilitate accurate positioning of the probe 125 into the desired tissue site. Moreover, the length of the guiding channels 143 may determine how deep a probe 125 may penetrate into tissue. For example, given the probes 125 in a probe assembly 120 have substantially the same length, the probe 125 inserted into a shorter guiding channel 143 may penetrate deeper into the tissue than the probe 125 inserted into a longer guiding channel 143.

The guide assembly 140 may include a suitable anchoring mechanism 149, such as, for example, pins, barbed projections, screws, or adhesives, to secure the guide assembly 145 and the probe assembly 120 onto the tissue surface. The anchoring mechanism 149 can be a permanent attachment or permit removal from the tissue surface. In some exemplary embodiments, the anchoring mechanism 149 may include additional electrodes and may anchor into tissue or any structure (e.g., bone) near tissue.

The guide assembly 140 may have a greater number of guiding channels 143 than the number of probes 125 of the probe assembly 120 and, therefore, some of the guiding channels 143 may not be used. The guide assembly 140 may include many different patterns so as to receive many different types of probe assemblies 120. In an exemplary embodiment, a single guiding channel may be configured to accommodate multiple probes. In another exemplary embodiment, the housing of a guide assembly may be comprised of multiple pieces.

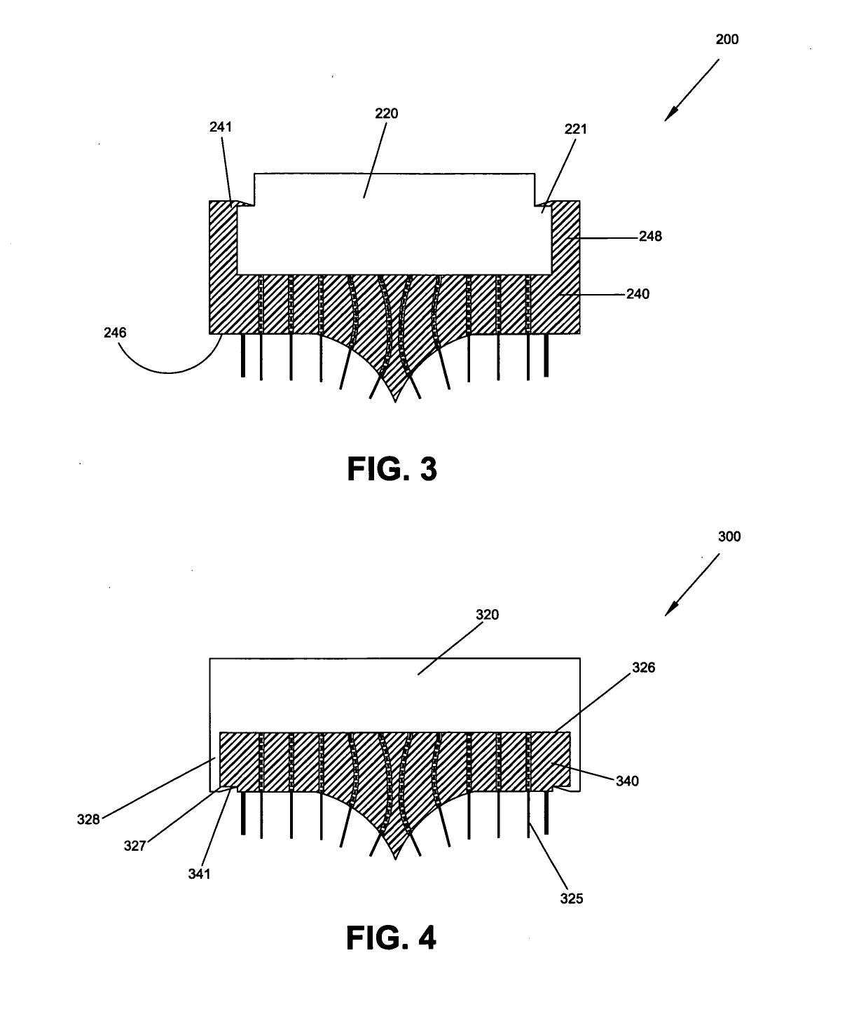

According to some exemplary embodiments, a probe assembly 220, 320 and a guide assembly 240, 340 may be configured to detachably or permanently engage with one another. For example, as shown in FIG. 3, the guide assembly 240 may define a housing or recess 246 configured to receive the probe assembly 220 therein. To facilitate the holding of the probe assembly 220 in the recess 246, the guide assembly 240 may include a holding flange 247, flap, or claw extending from and/or around an inside surface near its top surface. The sides 248 of the guide assembly 240 may have sufficient flexibility to bend and thereby permit insertion of the probe assembly 220 until a step 221 of the probe assembly 220 is engaged by the flange 247. In some exemplary embodiments, the sides 248 may be sufficiently rigid so that the sides 248 in combination with the flanges 247 may function as a guide rail configured to slidably receive the probe assembly 220 in a lateral direction.

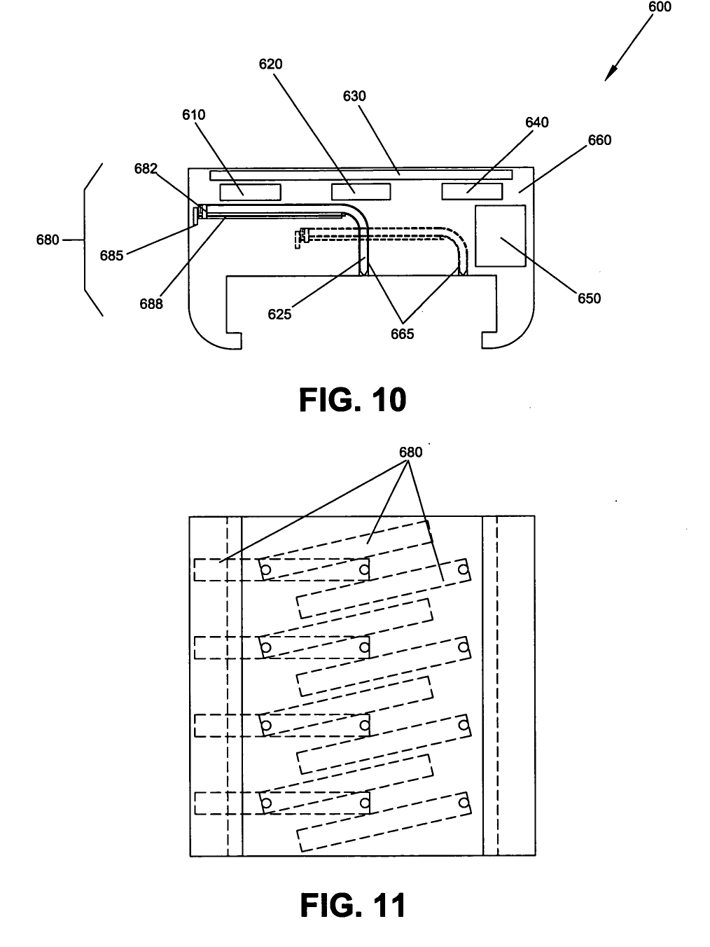

In an alternative embodiment, as shown in FIG. 4, the probe assembly 320 may form a recess 326 configured to receive the guide assembly 340 therein. Similar to the embodiment shown in FIG. 3, to facilitate the holding of the guide assembly 340 in the recess 326, the probe assembly 320 may include a holding flange 327, flap, or claw extending from and/or around an inside surface near its bottom surface. The sides 328 of the probe assembly 320 may have sufficient flexibility to bend and thereby permit insertion of the guide assembly 340 until a step 341 of the guide assembly 340 is engaged by the flange 327. In some exemplary embodiments, the sides 328 may be sufficiently rigid so that the sides 328 in combination with the flanges 327 may function as a guide rail configured to slidably receive the guide assembly 340 in a lateral direction. An example of such a guide rail is shown in FIGS. 10 and 11. Any other engagement mechanism, such as, for example, snap-fasteners, screws, magnets, glues, and adhesives, may be used alternatively or additionally.

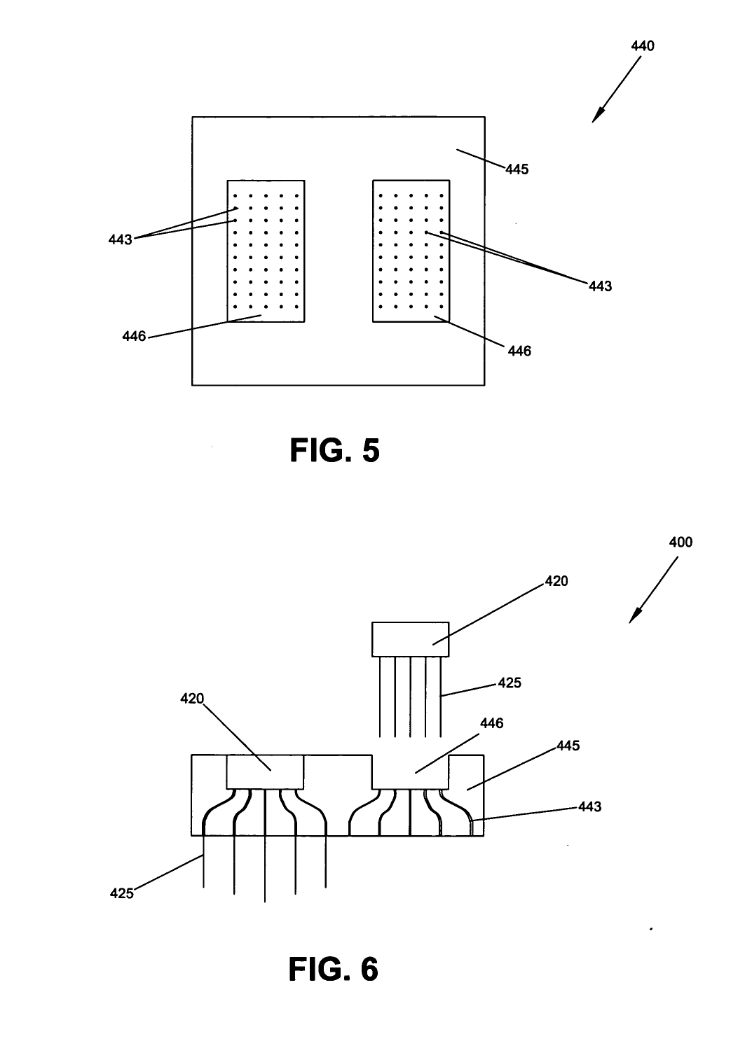

In another exemplary embodiment, a guide assembly 440 may be configured to receive a plurality of probe assemblies 420. For example, as shown in FIGS. 5 and 6, the housing 445 of the guide assembly 440 may include a plurality of recesses 446 for receiving the plurality of probe assemblies 420. In each of the recesses 446, the guide assembly 440 may define a plurality of guiding channels 443, each corresponding to each of the probes 425 of the probe assembly 420. In an alternative embodiment, a probe assembly may mate with a plurality of guide assemblies.

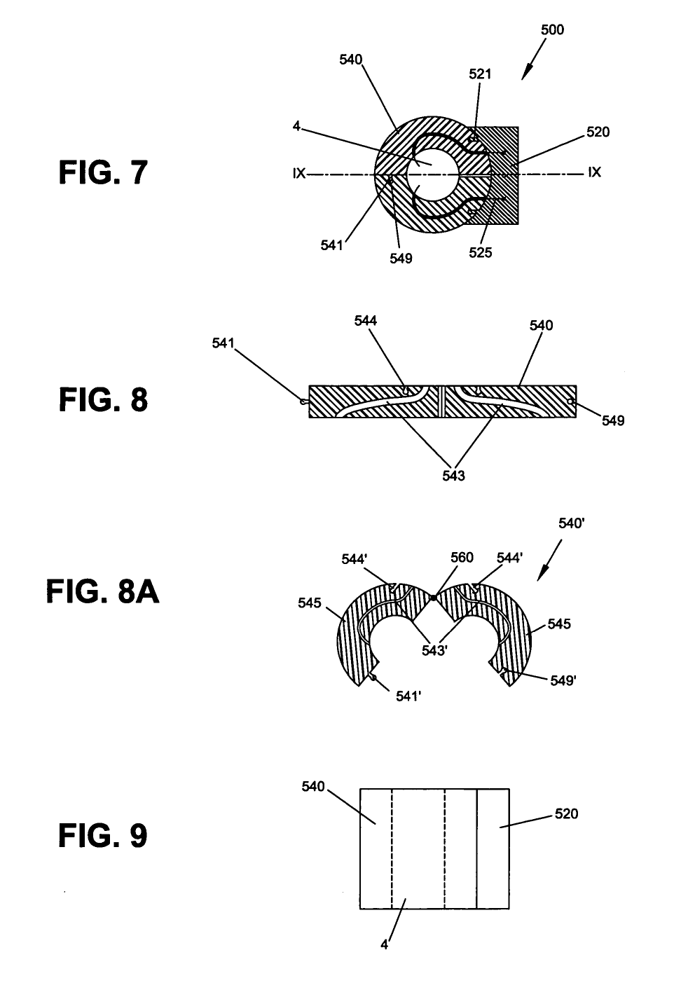

In still another exemplary embodiment, a guide assembly 540 may form a tubular configuration having a hollow inside space 4, as shown in FIGS. 7-9, so that tissue (e.g., a peripheral nerve cell) may be received in the hollow space 4. Similar to the exemplary embodiments described above, the guide assembly 540 may define a plurality of guiding channels 543 to guide the probes 525 to exit towards the hollow space 4. Alternatively or additionally, the guiding channels 543 may be configured to guide the probes 525 to exit towards the outer surface of the tubular guide assembly 540.

The guide assembly 540 may be formed of a flexible plate member, as shown in FIG. 8, whose ends may be interconnected to form the substantially tubular configuration. For that purpose, the ends of the flat plate member may include a suitable interconnecting mechanism, such as, for example, a snap-fastening projection 541 and a corresponding receiving hole 549. The bottom surface of the probe assembly 520 may have a suitable surface contour to match the corresponding contour of the guide assembly 540. The probe assembly 520 and the guide assembly 540 may be configured to engage with each other via a suitable attachment mechanism, such as, for example, a snap-fastening projection 521 and a corresponding receiving hole 544.

In an alternative embodiment, as shown in FIG. 8A, the guide assembly 540′ may be made of a relatively rigid material and comprise two substantially semi-circular portions 545 rotatably connected together with a hinge 560 (e.g., a live hinge). Similar to the guide assembly 540 shown in FIG. 8, the semi-circular portions 545 may define at least one guiding channel 543′ to guide the probes 525 to exit towards the hollow space 4. In an open configuration, as shown in the figure, the inner surface of the semi-circular portions 545 can be placed around a substantially tubular portion of tissue and, in a closed configuration, the semi-circular portions 545 may be subsequently closed to surround the tissue. The semi-circular portions 545 may be provided with a suitable interconnecting mechanism (e.g., a snap-fastening projection 541′ and a corresponding receiving hole 549′) to interconnect their ends to form the substantially tubular configuration. Also, the semi-circular portions 545 may include a suitable attachment mechanism (e.g., a snap-fastening projection 521′ and a corresponding receiving hole 544′) to engage the probe assembly 520.

As mentioned above, probes in a probe assembly may be movable in and out of the housing of the probe assembly. For that purpose, for example, a probe assembly 600 may comprise a probe deployment mechanism configured to advance and/or retract the probes 625 in and out of the housing 660. For example, as shown in FIGS. 10 and 11, the housing 660 may comprise internal guiding lumens 665, each configured to receive a probe 625, and, in each of the guiding lumens 665, a linear drive assembly 680 may be positioned to advance and/or retract each probe 625 in and out of the internal guiding lumen 665. In some exemplary embodiments, a single drive assembly 680 may be configured to deploy multiple probes 625 simultaneously. Each of the guiding lumens 665 may include one or more traces that may frictionally engage the corresponding probe for transmitting and/or receiving power or electrical signals, for example. The plurality of guiding lumens 665 may be coated and/or lubricated with a suitable material (e.g., Teflon) to reduce friction. In some exemplary embodiments, the guiding lumens 665 may be treated with a relatively hard (e.g., durable) material to substantially prevent damage to the lumens 665 by the probes 625. In some exemplary embodiments, a single drive assembly may control movement of multiple probes. In another exemplary embodiment, the housing 660 may be formed of multiple pieces.

Advancement and/or retraction of the probes 625 may be controlled automatically by the probe assembly, the array system, or other system component. Alternatively or additionally, the probes may be manually controlled by an operator. For example, a linear drive mechanism that may control one or more probes 625 may include an automatic controller or a remote controller. In some exemplary embodiments, upon actuation of a controller (e.g., pressing a button on a remote control), one or more probes 625 may be precisely deployed into tissue so as to place one or more electrodes at a predetermined depth. The electrodes may then detect signals at that tissue location, and the detected signals may then be reviewed. The review of the signals, or lack of signals, may be performed either automatically by the system or manually by the operator. If the detected signals are adequate for the intended purpose (e.g., above a threshold level or with a minimum modulation rate), the positioning process may end, and the probes 625 may remain at that location. If, on the other hand, the detected signals are inadequate for the intended purpose (e.g., below a threshold level or below a minimum modulation rate), the probes 625 may be automatically or manually advanced or retracted from that location until adequate signals may be detected by the probes 625.

Alternatively or additionally, the system may transmit stimulating signals (e.g., energy such as stimulating or polarizing energy or delivery of drug) to the probes 625 and, simultaneously or subsequently, detect and review for adequate response, such as adequate signals received from the tissue, or an acceptable physiologic response, such as an acceptable clinical outcome expected from an associated therapy (e.g., prevention or reduction of an epileptic seizure by delivery of stimulating energy to nerves or other tissue, or improvement in motor function of a stroke patient by applying an electromagnetic field to the patient's brain). If the response is not adequate or if a level of improvement is desired, the probes may be repositioned automatically or manually, and the process may be repeated until an adequate response is detected or a lack of improvement is confirmed. In an exemplary embodiment, quantitative thresholds may be imbedded in one or more components such that measured responses can be compared to these thresholds during the process of optimizing probe position. The positioning and/or repositioning of the probes 625 may be performed during or after (e.g., hours, days, or months) implantation of the probe assembly carrying the probes 625 within a body. In some exemplary embodiments, a sub-optimal position of probe deployment may be autonomously detected by a system component, and the probe 625 may be advanced and/or retracted automatically without operator intervention.

In the exemplary embodiment shown in Fig.10, the linear drive assembly 680 may comprise a lead screw 688 extending along a substantial portion of the guiding lumen 665, a clamp 682 configured to engage the probe 625 and the lead screw 668, and a stepper motor 685 configured to drive the clamp 682 so as to move the probe 625 along the lead screw 688. Due to its limited space within the housing 660, the guiding lumens 665 and the corresponding linear drive assemblies 680 may be positioned at different levels (e.g., at different depths within the housing 660) and/or may be extended at different angles, as shown in FIGS. 10 and 11. It should be understood that various exemplary embodiments of a linear drive assembly shown in FIGS. 10-20 may be configured to deploy multiple probes 625 simultaneously or only a single probe 625 at a time.

The probe assembly 600 may be a Micro Electro-Mechanical system (MEMS) integrating various mechanical elements, motors, actuators, sensors, and/or electronics in a common silicon substrate or any other applicable substrate (e.g., a semiconductor substrate). By utilizing advanced microfabrication techniques, MEMS technology enables production of the probe assembly 600 having miniaturized electro-mechanical features in the range of nanometers to millimeters.

In addition to the linear drive assembly 680, the probe assembly 600 may also include a memory storage device 610, a signal processing unit 620 (e.g., including appropriate signal processing circuitry for performing one or more of amplifying, filtering, sorting, conditioning, translating, interpreting, encoding, decoding, combining, extracting, sampling, multiplexing, analog to digital converting, digital to analog converting, mathematically transforming, and/or otherwise processing multicellular signals), an inductive power transfer device 630, a wireless transceiver device 640 (e.g., radiofrequency, infrared, ultrasound, and/or microwave communication module), and/or a power supply 650 (e.g., rechargeable battery or capacitor).

In an exemplary embodiment, the probe assembly 600 may comprise a CPU and/or a microcontroller element as well as a memory storage device for automatically performing one or more functions (e.g., a function dependent on the signals received by one or more electrodes of the probe assembly 600). In another exemplary embodiment, the probe assembly 600 may comprise one or more of: a power transfer device such as a device that may receive electromagnetic energy from a coil external to the patient's body; a power conversion device such as a device that may convert non-electrical energy to electrical energy; a wireless communication device; a drug delivery assembly or reservoir; an electromagnetic field generator; a light source; a camera assembly; an impedance measurement device such as an impedance device configured to determine the impedance of one or more electrodes or the impedance of the patient's tissue between two or more electrodes; a radiopaque marker; and a power supply such as a capacitor.

The probe assembly 600 may also include, but not be limited to: drug delivery assembly; an EM field generator (e.g., for agent delivery using electroporation or iontopheresis); a light producing element (e.g., UV source for infection control); a photo detector element; a camera or other visualization assembly (e.g., fiber optic, lens, etc.) for, e.g., confirming position and attachment status; a physiologic sensor; a chemical sensor; a motion sensor; a blood sensor (e.g., blood glucose sensor); a temperature sensor; a pressure sensor; an impedance measurement assembly; a volume sensor; a heating/cooling element; a stimulator (e.g., electrical stimulator or a mechanical vibrator); a force sensing assembly (e.g., strain gauge or accelerometer); and a radiopaque marker.

The probe assembly 600 also may include an embedded identification, such as, for example, an RF TAG or another embedded unique code that may be transmitted to another device (e.g., processing unit) independently or in combination with the cellular signals. This embedded identification may enable the system to identify the specific component, when, for example, there are multiple components within the system and/or multiple patients using the systems.

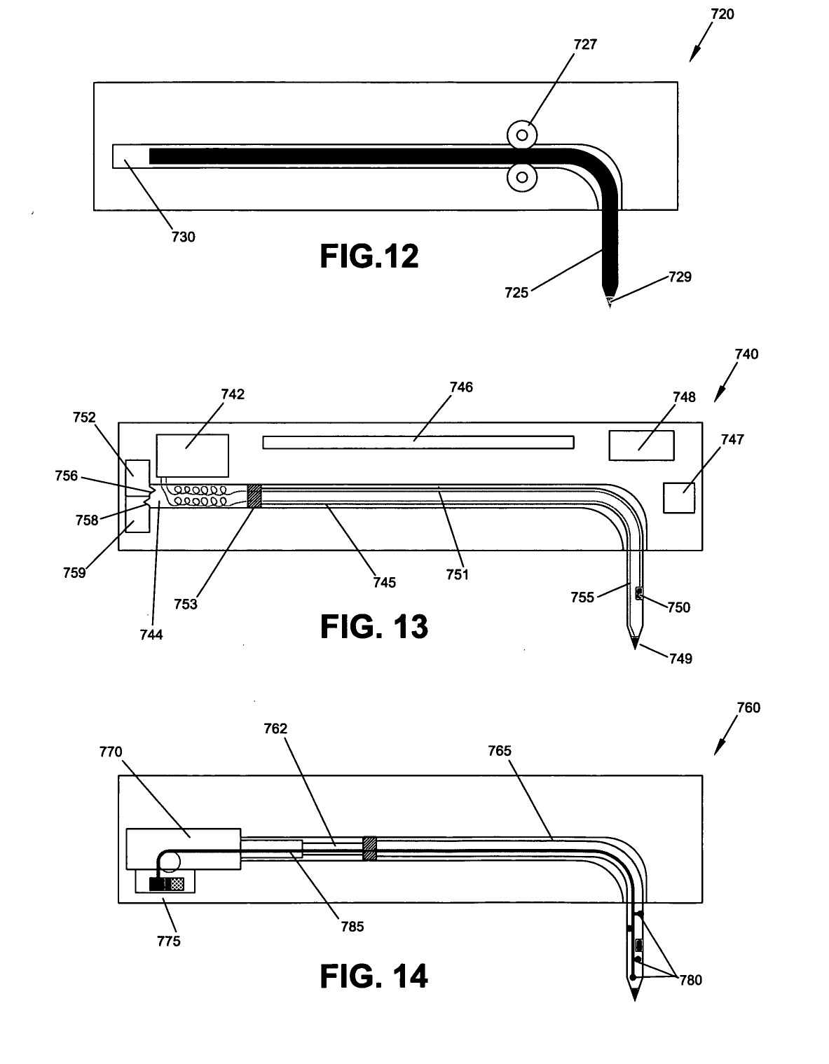

FIG. 12 shows a linear drive assembly 720 having one or more pinch rollers 727, according to an exemplary embodiment consistent with the invention. The pinch rollers 727 may be positioned in a fixed location within or adjacent the guiding lumen 730 in contact with a surface of the probe 725. Rotating the rollers 727 counterclockwise or clockwise may cause the probe 725 to move distally or proximally, respectively, along the guiding lumen 730. In some of the preferred embodiments, the pinch rollers 727 may be driven by one or more MEMS motors for directly driving the rollers 727. As shown in the figure, the probe 725 may include a distal element 729 (e.g., electrode or other functional element) at its distal tip. The distal element 729 may be connected to one or more conduits (e.g., wires or traces, optical fibers, etc.) and may be used to record cellular signals, send energy to tissue, or perform other various functions. In some exemplary embodiments, the probe 725 may include various types of function modules including, but not limited to: recording electrodes, stimulating electrodes; light emitting assemblies (e.g. photo or laser diode); light receiving assemblies (e.g. phototransistors or other photosensors); drug delivery probes; magnetic field generators; heating/cooling probes; or any other function modules known in the art.

According to another exemplary embodiment, FIG. 13 shows a linear drive assembly 740 utilizing discharging gas to move the probe 745. The assembly 740 may include a gas discharging member 752 (e.g., electrolytic cell) having an outlet valve 756 and a gas suction member 759 (e.g., vacuum source) having an inlet valve 758. The proximal end of the probe 745 may include a suitable sealing member 753 so that the space inside the guiding lumen 744 between the inlet and outlet valves 756, 758 and the sealing member 753 within the guiding lumen 744 may have a fluid tight seal, like a plunger in a piston cylinder. To advance the probe 745 distally out of the guiding lumen 744, the outlet valve 756 may be opened to discharge gas into the guiding lumen 744, such as gas created through activation of an electrolytic cell, not shown. The pressure increase caused by the gas discharge may cause the probe 745 to move distally. To retract the probe 745 proximally, the inlet valve 758 may be opened and the gas suction member 759 may be actuated to suck the gas discharged into the guiding lumen 744. The pressure decrease caused by the suction member 759 may retract the probe 745 proximally.

FIG. 13 also shows that the probe 745 may comprise two electrodes 749, 750. Each of the electrodes 749, 750 may be connected to a signal processor 742 via a cable 755, 751 to transmit signals detected by the electrodes 749, 750. The drive assembly 740 may be a stand-alone system including a power transfer device 746, a wireless transmitter 748, and an energy storage device 747, thus allowing numerous signal processing tasks to be performed by the probe 745 and possibly eliminating a multi-conductor cable connection for transmitting the detected signals or a processed version of the detected signals to a separate device. Additional functional elements, such as memory storage, microcontroller, and/or CPU functions, may be included in the probe 745.

FIG. 14 shows another exemplary embodiment of a linear drive assembly 760 having a hydraulic piston drive mechanism to drive the probe 765. The assembly 760 may include a suitable hydraulic piston assembly 770 configured to drive a telescoping piston 762 that may be connected to the proximal end of the probe 765. The probe 765 shown in FIG. 14 may include one or more fluid reservoirs and/or ports 780 for storage and/or delivery of therapeutic agents or drugs. The therapeutic agents or drugs may be supplied to the fluid ports 780 via a pump 775 and a drug delivery tube 785. In an exemplary embodiment, the drive assembly 760 may be refillable with additional or alternative agents by a suitable refill mechanism. In another exemplary embodiment, the ports 780 may utilize iontophoresis techniques, such as one or more electromagnetic fields generated by probe 765, to distribute or propel the therapeutic agent into the tissue neighboring ports 780.

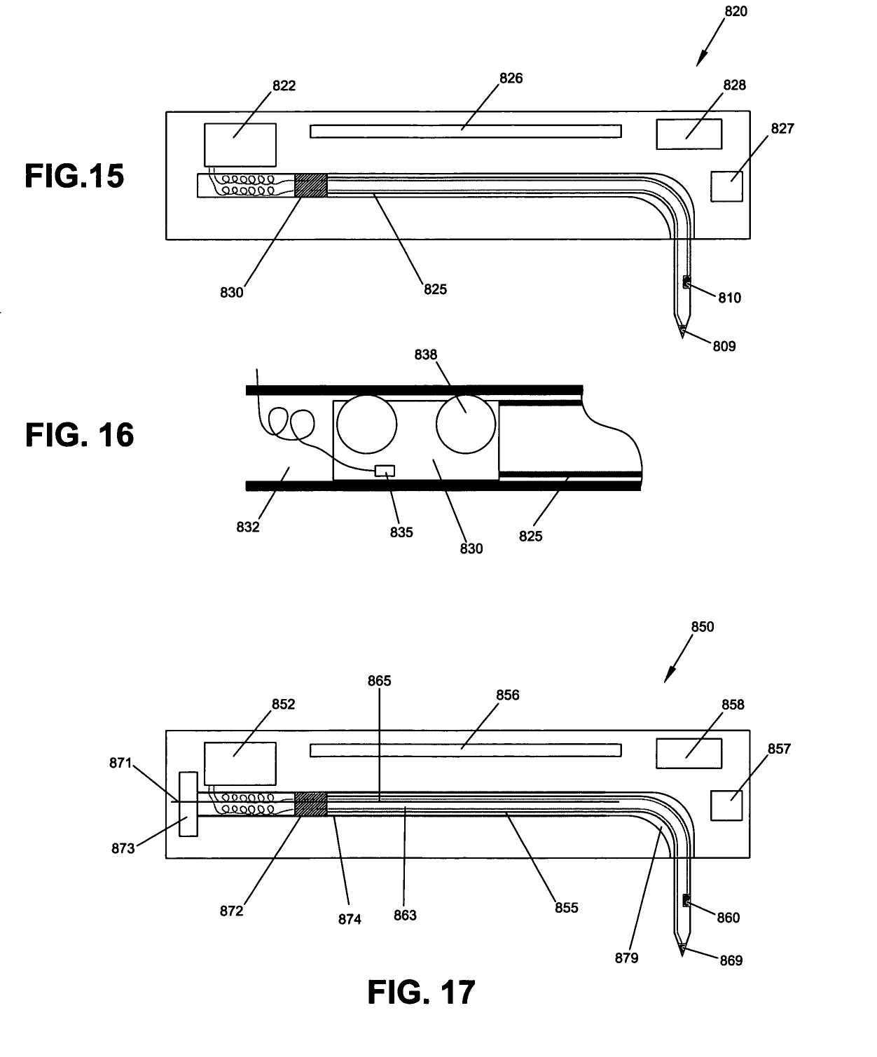

FIGS. 15 and 16 show still another exemplary embodiment of a linear drive assembly 820 having an inch-worm drive mechanism 830. The inch-worm drive mechanism 830 may comprise one or more rollers 838 coupled to the proximal end of the probe 825 and an electronic control module 835 configured to control rotational direction and speed of the rollers 838. The rollers 838 may contact the inner surface of the guiding lumen 832 so that, when rollers 838 rotate, the probe 825 may move together with the rollers 838. Similar to the embodiment described above with reference to FIG. 13, the drive assembly 820 shown in FIG. 15 may be a stand-alone system including a signal processor 822 connected to the electrodes 809, 810 via suitable cables, a power transfer device 826, a wireless transmitter 828, and an energy storage device 827. While the two rollers 838 are shown on the same side of the drive mechanism 830, in an alternative embodiment, one or more rollers 838 may be placed on each side of the drive mechanism 830.

According to another exemplary embodiment of a linear drive assembly, as shown in FIG. 17, the guiding lumen 879 may include an inner tube 874 having inner threads, and the proximal portion of the probe 855 may include a screw 872 that may be configured to engage with and ride over the inner threads of the inner tube 874. The inner tube 874 may be rotatable with respect to the guiding lumen 879, and the drive assembly 850 may include a stepper motor 873 configured to rotate the inner tube 874. When the stepper motor 873 is actuated to rotate the inner tube 874, the screw 872 together with the probe 855 may move distally or proximally, depending on the rotational direction of the inner tube 874, along the guiding lumen 879. The drive assembly 850 may include a locking mechanism for preventing the inner tube 874 to rotate with respect to the guiding lumen 879. For example, the drive assembly 850 may include an anti-rotation rod 871 that may be disposed slightly off-centered within the probe 855 and may be locked with the stepper motor 873. Due to its off-centered position, when the rod 871 is locked with the stepper motor 873, the rotation of the inner tube 874 with respect to the guiding lumen 879 may be prevented.

Similar to the embodiments described above with reference to FIGS. 13 and 15, the drive assembly 850 shown in FIG. 17 also may be a stand-alone system including a signal processor 852 connected to the electrodes 860, 869 via suitable cables 863, 865, a power transfer device 856, a wireless transmitter 858, and an energy storage device 857.

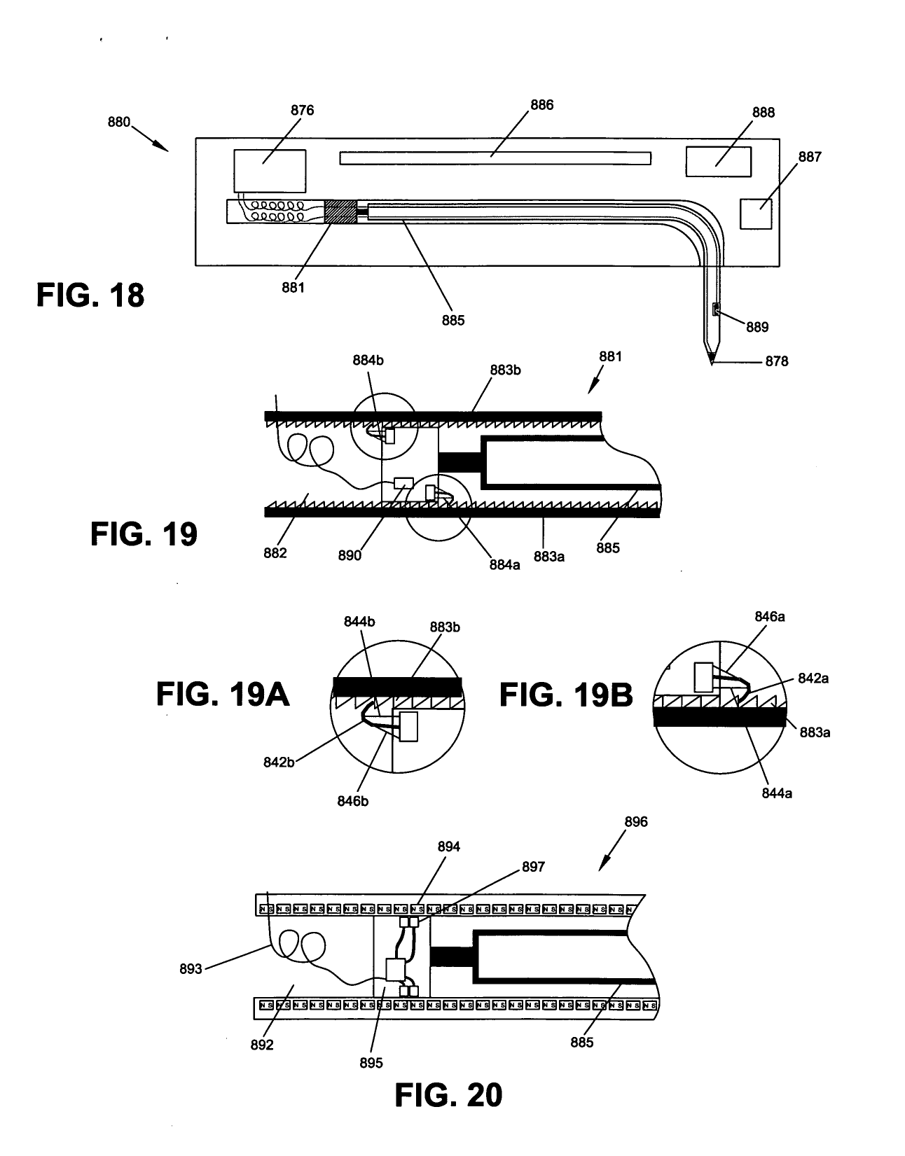

According to still another exemplary embodiment, a linear drive assembly 880 may utilize characteristics of a shape memory material. For example, as shown in FIGS. 18 and 19, the drive assembly 880 may comprise a memory wire drive mechanism 881 having a forward-moving member 884a and a backward-moving member 884b, each generally facing in the opposite direction from one another. In some exemplary embodiments, the linear drive assembly 880 may include only one of the forward- and backward-moving members 884a, 884b. The guiding lumen 882 may comprise at least two rows of teeth 883a, 883b formed on its inside surface that are configured to selectively engage with either the forward-moving member 884a or the backward-moving member 884b. For example, the teeth 883a, 883b in each row may have a shape (e.g., right-triangular cross-section) engageable with only one of the forward-moving member 884a and the backward-moving member 884b.

As best shown in FIGS. 18A and 18B, each of the moving members 884a, 884b may comprise a resiliently biased, flexible hook 842a, 842b and a pull wire 844a, 844b attached to a portion of the hook 842a, 842b. The hook 842a, 842b may be made of a spring material or an elastic or super-elastic metal alloy. The hook 842a, 842b may be attached to the pull wire 844a, 844b in such a way that contraction of the pull wire 844a, 844b causes the hook 842a, 842b to bend and engage one of the respective teeth 883a, 883b, applying a force against the tooth 883a, 883b so as to pull the probe forward (i.e., when the pull wire 844a of the forward-moving member 884a is contracted) or backward (i.e., when the pull wire 844b of the backward-moving member 884b is contracted). When the pull wire 884a, 884b is not contracted (e.g., released or rest state), the hook 842a, 842b due to its resilient bias force may restore its original shape to engage the next tooth. The same steps discussed above may be repeated to further move the probe forward or backward.

In some exemplary embodiments, the pull wire 844a, 844b may be made of a shape memory material, such as Nitinol wire, and may be biased to form a straight wire. Under a predetermined condition (e.g., elevated temperature or electric current), the wires 884a, 884b may bend or decrease in length to pull the hook 842a, 842b to bend and apply a pulling force again one of the teeth 883a, 883b. For example, upon subjecting the pull wire 844a of the forward-moving member 884a to the predetermined condition, the wire 844a may bend to engage the teeth 883a, pulling itself forward with respect to the teeth 883a, thereby moving the probe 885 forward. Likewise, upon subjecting the pull wire 844b of the backward-moving member 884b to the predetermined condition, the wire 844b may bend to engage the teeth 883b so as to move itself backward with respect to the teeth 883b. Alternatively, the pull wire 844a, 844b may be biased in a contracted state so as to cause the hook 842a, 842b to engage one of the two rows of teeth 883a, 883b and, under a predetermined condition, the pull wires 844a, 844b may expand to cause the hook 842a, 842b to disengage from the teeth 883a, 883b. Therefore, by selectively actuating one of the forward-and backward-moving members 884a, 884b, the probe 885 coupled to the memory wire drive mechanism 881 may be moved distally or proximally along the guiding lumen 882. In an alternative embodiment, the pull wire 844a, 844b may be mechanically pulled and/or pushed to achieve the same operational effect.

In an exemplary embodiment, the moving members 884a, 884b may include a release wire 846a, 846b configured to disengage the hook 842a, 842b from the teeth when the probe 885 moves distally or proximally. For example, when the probe 885 is moving proximally in the backward direction, the release wire 846a in the forward-moving member 884a may be configured to contract and pull the hook 842a so as to disengage the hook 842a from the teeth 883a. Similarly, when the probe 885 is moving distally in the forward direction, the release wire 846b in the backward-moving member 884b may be configured to contract and pull the hook 842b so as to disengage the hook 842b from the teeth 883b. In some exemplary embodiments, the release wire 846a, 846b may be made of a shape memory material, such as Nitinol wire, and may also have a resilently biased shape.

The exemplary embodiment shown in FIG. 18 is also a stand-alone system, like the systems of FIGS. 13, 15, and 17. The drive assembly 880 may include a signal processor 876 connected to the electrodes 878, 889 via suitable cables, a power transfer device 886, a wireless transmitter 888, and an energy storage device 887.

FIG. 20 shows another exemplary embodiment of a linear drive assembly 896 utilizing an electromagnetic drive mechanism. As shown in the figure, the guiding lumen 892 may include a series of magnets 894, and the drive mechanism may include one or more electro-magnets 897 controllable by an electronic module 895 connected to a power and control cable 893. Selectively supplying current to the electro-magnets 897, individually or in combination, in a forward or reverse direction, creates magnetic fields that react with the magnetic fields of the permanent magnets 894 and, thereby, cause the drive mechanism to advance or retract in small, highly precise steps. The magnitude of advancement and/or retraction may be determined by the geometric location between the sets of magnets 894, 897. Once the appropriate electromagnet is energized, “like-pole” magnetic fields produce a repulsive force causing the drive mechanism to move. When the electromagnet 897 approaches a permanent magnet 894 with a dissimilar pole, the resultant attractive force can be used to continue motion and/or prevent over-travel of the drive mechanism. Selective energizing of one or more electromagnets 897 may be used to cause the drive mechanism to move in discrete steps, in a forward or backward direction, by creating repulsive and attractive forces with the associated permanent magnets 894. In an alternative embodiment, the magnets 894 may be electromagnets connected to a controller, and magnets 897 may be permanent magnets. In an exemplary embodiment, a linear position indicator may be included, such as a resistive strip placed along the trajectory that makes contact with a conductive wiper element integral to the module 895, wiper element, and resistive strip (not shown). The information received from the linear position indicator can be used to confirm proper advancement or retraction of the probe 885. In the condition where appropriate advancement or retraction of the probe 885 is not achieved when electromagnets 897 are energized at a first energy level, an increase in energy may be supplied until adequate motion is confirmed. In this configuration, the energy supplied to electromagnets 897 can be minimized, and increased as appropriate, making drive assembly 896 extremely power efficient.

As is apparent, various exemplary embodiments of the linear drive mechanism described above may permit very minor, accurate adjustment of each probe, which in turn may permit very precise positioning and adjustment of the probes within the tissue of interest.

Although certain features of the drive assembly and/or the probe are discussed with only a particular embodiment above, it should be understood that any combination of various features may be incorporated into or used with any other embodiments discussed above.

FIG. 21 shows an electrode array system 300 implanted on a patient's brain 50 inside the skull 60, in particular on a crevice of the brain 50. The system 300 is substantially similar to the exemplary embodiment described with reference to FIG. 4 and, thus, detailed description of the system 300 is omitted. As shown in FIG. 21, the tissue sites into which some of the probes 325 are to be placed may be located on a side surface of a brain crevice. In that case, the probes having substantially straight configurations may not accurately access those desired tissue sites. As mentioned above, the curved guiding channels of the guide assembly 340, in combination with the tissue contacting surface 341 closely matching the topography of the tissue surface, may enable the probes 325 to penetrate into tissue sites that may otherwise be inaccessible for straight probes, thereby facilitating accurate positioning of the probes 325 into the desired tissue site. In an exemplary embodiment, the tissue contacting surface 341 may be manufactured based on specific patient information, such as brain topography information generated during an MRI procedure.

To allow for a larger sized system 300 (e.g., a stand-alone system including energy storage, signal processing, and wireless transmission device) to be placed inside the cranium of the patient, a receiving recess 61 may be surgically formed on the inner surface of the skull 60 to receive at least a portion of the electrode array system 300, as shown in FIG. 21. The recess may be formed by any suitable, known method. For example, bone cutting tools may be used to create grooves and recesses in the skull. In an exemplary embodiment, a bone portion may be removed from the skull while the system 300 is being placed and may be cut to form the recess 61 prior to being replaced to and reconnected to the skull 60.

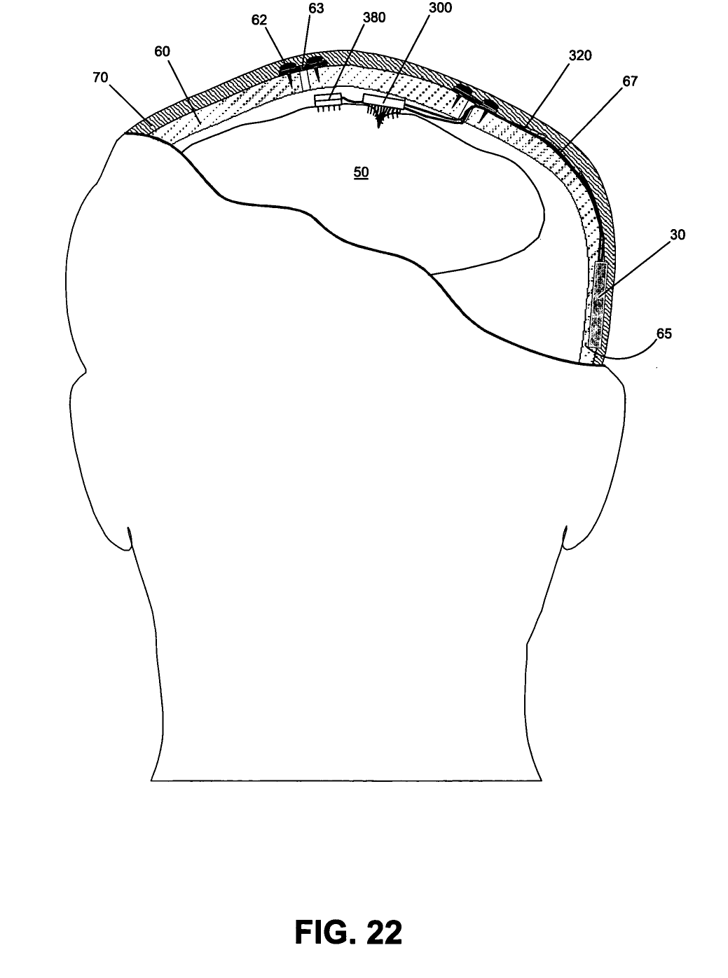

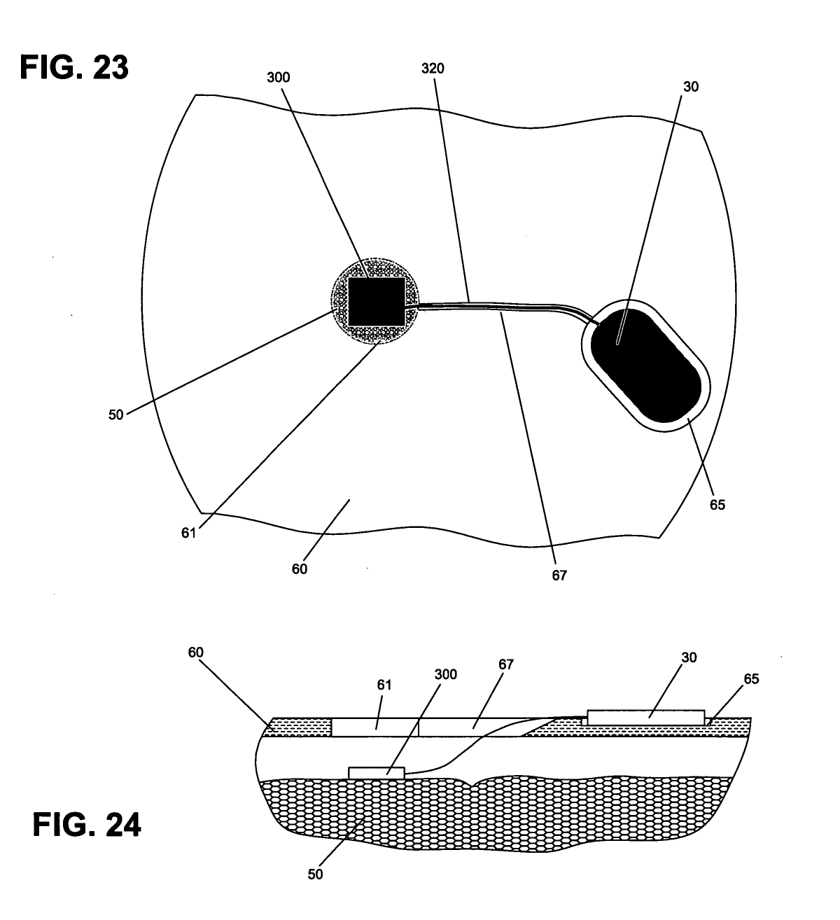

FIG. 22 generally illustrates a brain implant apparatus consistent with an embodiment of the invention, and FIGS. 23 and 24 illustrate an exemplary surgical method of placing the brain implant apparatus. As shown in these figures, the system includes one or more electrode array systems 300, 380 inserted into a patient's brain 50 (e.g., the cerebral cortex) through an opening 61 in the skull 60 (e.g., through an opening created by the removal of a bone flap during a procedure known as a craniotomy). As shown in FIG. 22, the electrode array systems 300, 380 may be identical or different, and may be placed in any location of the patient's brain 50 to detect electrical brain signals or impulses.

After the array systems 300, 380 are inserted into the patient's brain 50, a prosthetic plate and/or the original portion or sub-portion of the skull 60 (e.g., bone flap) removed during the craniotomy may be placed in the opening 61 in the skull 60 and attached with one or more surgical straps and/or bone screws 62, preferably with one or more attaching straps 63. It may be desirable that all implanted components avoid the need to protrude through the skin of the patient, such as for cosmesis and reduced infection risk. Thus, the processing unit 30 may be placed in a recess 65 in the top of the skull 60 created during the same surgical procedure, at a location near and above the ear of the patient as shown, or at another location under the scalp.

The apparatus may comprise a processing unit 30 that may be in close proximity to the array systems 300, 380 (e.g., less than 20 cm between the processing unit 30 and the array systems 300, 380). For example, the processing unit 30 may be implanted under the skin of the patient, such as, for example, on top of the skull 60 of the patient under the scalp 70, as shown in FIG. 24. A wire bundle 320, single or multi-conductor cable (e.g., electrical wires and/or optical fibers), may connect between one or more array systems 300, 380 and the processing unit 30. The wire bundle 320 may be received in an elongated slit 67 or slot that may be surgically created on the outer surface of the skull 60 extending between the opening 61 and the recess 65 of the skull 60, as shown in FIG. 23.

FIGS. 23 and 24 also illustrate a unique method of implanting an electrode array system into a patient's brain. The method may include making a small opening 61 (e.g., “burr hole”) in the skull 60, which may only be slightly larger than the planar dimension of the array 300. Due to the limited size of the opening 61, it may be difficult to place the array 300 having wires or wire bundles 320 into the opening 61. Moreover, even if the array 300 having wires or wire bundles 320 are properly placed in the brain, the wires or wire bundles 320 are likely to have sharp bends when they exit through the opening 61, which may be highly undesirable. Thus, the slit 67 in the skull 60 may allow the wire bundle 320 to avoid sharp turns, as best shown in FIG. 24. The length of the slit 67 may be chosen such as to provide smooth transition between the array location and the location of the processing unit 30. The slit 67 may extend completely through the skull, such as at the location proximate opening 61, and may transition to penetrating partially into the top of the skull (scalp side) without penetrating through the skull (to the brain side) as slit 67 approaches recess 65. By way of example only, the opening 61 may be as little as 1˜2 cm in diameter, and may be closed with an artificial plug or bone material after the implant procedure.

The wire bundle 320 may include other conductors or conduits such as a conductor that provides a reference signal at a location in proximity to the electrodes, a fiber optic cable used to receive images from the implantation location such as a fiber optically connected to one or more lenses mounted on an external surface of the array systems 300, 380, or a fluid flow tube for delivering a drug to the array system 300. Alternatively or additionally, the processing unit 30 and the array systems 300, 380 may communicate via a wireless communication module.

In some exemplary embodiments, individual probes 325 may be attached each to individual conductors of the wire bundle 320, and the wire bundle 320 may include at least two conductors that do not attach to the probes 325 but are placed to provide relevant reference signals for one or more signal processing functions. By way of example only, the conductive wires of the wire bundle 320 may-have a diameter of approximately 25 μm and may comprise a blend of gold and palladium. The wire bundle 320 and the processing unit 30 may be sealed such that the signals, conductive surfaces, and other internal components of the wire bundle 320 and the processing unit 30 may be appropriately protected from contamination by body fluids and other contaminants. In an exemplary embodiment, the wire bundle 320 may be a flex or ribbon cable and may include an attachment member at either or both ends.

The processing unit 30 may include an appropriate module for amplifying the cellular signals (e.g., with a gain of approximately one hundred, a working frequency range of about 0.001 Hz to about 7.2 kHz, a power requirement of approximately 1.6 V, and a power dissipation of approximately 30 mW). The processing unit 30 may further include additional signal processing circuitry to perform one or more functions including, but not limited to: filtering, sorting, conditioning, translating, interpreting, encoding, decoding, combining, extracting, sampling, multiplexing, analog to digital converting, digital to analog converting, mathematically transforming, and/or otherwise processing multicellular signals to, for example, generate a control signal for transmission to a controlled device. The processing unit 30 may transmit the control signal through an integrated wireless communication module, such as, for example, radiofrequency communications, infrared communications, inductive communications, ultrasound communications, and microwave communications. This wireless transfer may permit the array systems 300, 380 and the processing unit 30 to be completely implanted under the skin of the patient, avoiding the need for implanted devices that may require exit of a portion of the device through the skin surface. The processing unit 30 may further include a coil that may receive power, such as through inductive coupling, on a continual or intermittent basis from an external power transmitting device. This integrated coil or a separate coil may be used to transmit information in addition to or in place of power transmission. The power and information can be delivered to the processing unit 30 simultaneously such as through simple modulation schemes in the power transfer that may be decoded into information for processing unit 30 to operate.

The processing unit 30 may also transmit signals to one or more electrodes of the array systems 300, 380 so as to stimulate, polarize, or otherwise affect the neighboring nerves or other cells. Stimulating electrodes in various locations can be used by the processing unit 30 to transmit signals to the central nervous system, peripheral nervous system, other body systems, body organs, muscles and other tissue or cells. The transmission of these signals may be used to perform one or more functions including but not limited to: pain therapy, muscle or organ stimulation, seizure disruption, and patient feedback.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An electrode system comprising:
a probe assembly comprising a plurality of probes configured to penetrate tissue of a patient; and
a guide assembly comprising a plurality of guiding channels, each of the guiding channels being configured to guide one or more of the plurality of probes to a desired tissue site.

2. The system of claim 1, wherein at least one of the plurality of probes is configured to detect cellular signals.

3. The system of claim 1, wherein at least one of the plurality of probes is configured to deliver energy to tissue.

4. The system of claim 3, wherein the energy delivered to tissue is selected from the group consisting of: heat energy; cryogenic energy; light energy; radiation energy; chemical energy; mechanical energy; electrical energy; and any combination thereof.

5. The system of claim 1, wherein at least one of the probes is configured to deliver agent.

6. The system of claim 5, wherein the agent comprises a pharmaceutical agent.

7. The system of claim 1, wherein at least one of the probes is configured to produce a magnetic field.

8. The system of claim 1, wherein at least one of the probes comprises a sensor.

9. The system of claim 8, wherein the sensor is selected from the group consisting of: a thermal sensor; a pressure sensor; a chemical sensor; a force sensor; an electromagnetic field sensor; a physiologic sensor; a photodetector; a pH sensor; an oxygen sensor; a blood sensor; an electrode; and any combination thereof.

10. The system of claim 9, wherein the physiologic sensor comprises at least one of an electrocardiogram sensor and a blood glucose sensor.

11. The system of claim 1, wherein at least one of the probes is flexible.

12. The system of claim 1, wherein at least one of the probes is rigid.

13. The system of claim 1, wherein at least one of the probes has a resiliently biased shape.

14. The system of claim 13, wherein the resiliently biased shape has a curved portion.

15. The system of claim 1, wherein at least one of the probes comprises a shape memory material.

16. The system of claim 15, wherein the shape memory material comprises a shape memory alloy.

17. The system of claim 1, wherein at least two of the probes have lengths that are different from one another.

18. The system of claim 1, wherein at least two of the probes have thicknesses that are different from one another.

19. The system of claim 1, wherein at least one of the probes comprises a first functional element and a second functional element.

20. The system of claim 19, wherein at least one of the first and second functional elements comprises an electrode.

21. The system of claim 20, wherein the electrode is located at a distal tip of the probe.

22. The system of claim 19, wherein the first functional element is an electrode with a first set of characteristics, and the second functional element is an electrode with a second set of characteristics.

23. The system of claim 22, wherein the characteristics comprise at least one of: an impedance; a surface area; a material of construction; a surface texture; a porosity; a length; a width; a diameter; a thickness; a surface energy; a coating; and any combination thereof.

24. The system of claim 19, wherein at least one of the first and second functional elements comprises at least one of a photodiode and a photosensor.

25. The system of claim 1, wherein at least one of the probes or at least one of the guiding channels comprises a conductive trace.

26. The system of claim 25, wherein the conductive trace is configured to provide an electrical connection between the at least one of the probes and the at least one of the guiding channels.

27. The system of claim 1, wherein at least one of the probes comprises a lumen along at least a portion of its length.

28. The system of claim 27, wherein the lumen is configured to permit passage of fluid.

29. The system of claim 1, wherein the plurality of probes are arranged in an array.

30. The system of claim 1, wherein the probe assembly comprises a housing from which the plurality of probes project, and at least one of the plurality of probes is individually deployable from the housing.

31. The system of claim 30, wherein at least two of the plurality of probes are simultaneously deployable from the housing.

32. The system of claim 30, wherein the housing comprises a probe deployment mechanism configured to move the at least one of the plurality of probes relative to the housing.

33. The system of claim 30, wherein at least one of the probes is configured to be deployed while the housing is being implanted on the tissue of the patient.

34. The system of claim 30, wherein at least one of the probes is configured to be deployed after the housing is implanted on the tissue of the patient.

35. The system of claim 30, wherein the housing comprises at least one internal guiding lumen configured to receive at least one probe of the probe assembly, the housing comprising a drive assembly positioned adjacent the internal guiding lumen to move the probe within the internal guiding lumen.

36. The system of claim 35, wherein the drive assembly is controllable manually.

37. The system of claim 35, wherein the drive assembly is controllable remotely.

38. The system of claim 35, wherein the drive assembly is controllable automatically.

39. The system of claim 35, wherein the drive assembly comprises:
a screw extending along at least a portion of the internal guiding lumen;
a drive member configured to engage the probe and the screw; and
a drive mechanism configured to drive the drive member so as to move the probe distally or proximally along the internal guiding lumen.

40. The system of claim 35, wherein the drive assembly comprises at least one pinch roller in contact with the probe, wherein rotating the roller causes the probe to move distally or proximally along the internal guiding lumen.

41. The system of claim 40, wherein the at least one pinch roller is disposed adjacent the internal guiding lumen or has a portion disposed in the internal guiding lumen.

42. The system of claim 40, wherein the at least one pinch roller comprises two pinch rollers.

43. The system of claim 35, wherein the drive assembly comprises a gas discharging member having an outlet valve and being configured to discharge gas into the internal guiding lumen, wherein discharge of the gas causes the probe to advance the probe distally along the internal guiding lumen.

44. The system of claim 43, wherein the gas discharging member comprises an electrolytic cell.

45. The system of claim 43, wherein the drive assembly further comprises a gas suction member having an inlet valve and being configured to suction gas out of the internal guiding lumen, wherein suctioning of the gas causes the probe to retract proximally along the internal guiding lumen.

46. The system of claim 35, wherein the drive assembly comprises a suction member having an inlet valve and being configured to suction fluid out of the internal guiding lumen so as to retract the probe proximally along the internal guiding lumen.

47. The system of claim 35, wherein the drive assembly comprises:
an extendable piston having a distal end connected to the probe; and
a drive mechanism configured to extend or retract the extendable piston so as to move the probe distally or proximally along the internal guiding lumen.

48. The system of claim 47, wherein the drive mechanism comprises at least one of a hydraulic drive element and a pneumatic drive element.

49. The system of claim 35, wherein the drive assembly comprises:
a roller coupled to a portion of the probe, a surface of the roller being in contact with an inner surface of the internal guiding lumen; and
a controller configured to control rotation of the roller,
wherein rotation of the roller causes the probe to move distally or proximally along the internal guiding lumen.

50. The system of claim 49, wherein the drive assembly further comprises a second roller coupled to the probe.

51. The system of claim 35, wherein the drive assembly comprises:
a tube having inner threads and being disposed inside the internal guiding lumen;
a screw attached to a proximal end of the probe, the screw being configured to engage with and ride over the inner threads; and
a drive mechanism configured to rotate at least one of the tube and the screw,
wherein rotating at least one of the tube and the screw causes the screw to move relative to the tube so as to move the probe distally or proximally along the internal guiding lumen.

52. The system of claim 51, wherein the drive mechanism comprises a stepper motor.

53. The system of claim 35, wherein the drive assembly comprises:
first teeth disposed at least partially along the internal guiding lumen;
a drive member coupled to the probe, the drive member comprising a forward-moving member configured to engage or disengage at least one tooth of the first teeth when a first predetermined condition is applied, such that when the forward-moving member engages and disengages the at least one tooth of the first teeth, the probe moves distally along the internal guiding lumen; and
an actuator configured to actuate the forward-moving member so as to move the probe distally along the internal guiding lumen.

54. The system of claim 53, wherein the forward-moving member comprises a shape memory material.

55. The system of claim 53, wherein the forward-moving member is disengaged from the at least one tooth of the first teeth when the probe moves proximally.

56. The system of claim 53, wherein the drive assembly further comprises second teeth disposed at least partially along the internal guiding lumen, and wherein the drive member further comprises a backward-moving member configured to engage or disengage at least one tooth of the second teeth when a second predetermined condition is applied, so that when the backward-moving member engages and disengages the at least one tooth of the second teeth, the probe moves proximally along the internal guiding lumen.

57. The system of claim 35, wherein the drive assembly comprises:
teeth disposed at least partially along the internal guiding lumen;
a drive member coupled to the probe, the drive member comprising a backward-moving member configured to engage or disengage at least one tooth when a first predetermined condition is applied, such that when the backward-moving member engages and disengages the at least one tooth of the teeth, the probe moves proximally along the internal guiding lumen; and
an actuator configured to actuate the backward-moving member so as to move the probe proximally along the internal guiding lumen.

58. The system of claim 57, wherein the backward-moving member comprises a shape memory material.

59. The system of claim 57, wherein the backward-moving member is disengaged from the at least one tooth when the probe moves distally.

60. The system of claim 35, wherein the drive assembly comprises:
at least one first magnet disposed at least partially along the internal guiding lumen;
a drive member coupled to the probe and comprising at least one second magnet; and
a controller for energizing either the first magnet or the second magnet,
wherein energizing either the first magnet or the second magnet causes the probe to move distally or proximally along the internal guiding lumen.

61. The system of claim 60, wherein at least one of the first and second magnets is configured to be activated by the controller.

62. The system of claim 60, wherein at least one of the first and second magnets is an electromagnet.

63. The system of claim 60, wherein at least one of the first and second magnets is a permanent magnet.

64. The system of claim 60, wherein one of the first and second magnets is an electromagnet, and the other of the first and second magnets is a permanent magnet.

65. The system of claim 60, wherein at least one of the first and second magnets comprises a plurality of magnets.

66. The system of claim 60, wherein the controller is configured to energize either the first magnet or the second magnet by supplying electrical current to the magnet to be energized.

67. The system of claim 66, wherein the electrical current supplied in a first direction causes the probe to move distally along the internal guiding lumen, and the electrical current supplied in a second direction opposite the first direction causes the probe to move proximally along the internal guiding lumen.

68. The system of claim 60, wherein the at least one first magnet or the at least one second magnet comprises a plurality of magnets arranged in a row, the plurality of magnets separated from one another by a predetermined distance.

69. The system of claim 68, wherein energizing either the first magnet or the second magnet causes the probe to move a length that is substantially equal to the predetermined distance.

70. The system of claim 30, wherein the housing further comprises at least one of: a memory storage device; a signal processing unit; a power transfer device; a power conversion device; a wireless communication device; a CPU; a microcontroller; a drug delivery assembly or reservoir; an electromagnetic field generator; a light source; a camera assembly; an impedance measurement device; a radiopaque marker; and a power supply.

71. The system of claim 30, wherein the housing further comprises a power transfer device configured to convert non-electrical energy to electrical energy.

72. The system of claim 30, wherein the housing comprises a wireless communication device configured to transfer information via: radiofrequency; microwave; infrared; ultrasound; or any combination thereof.

73. The system of claim 30, wherein the housing further comprises a signal processing element configured to perform a signal processing function selected from the group consisting of: amplification; filtering; sorting; conditioning; translating; interpreting; encoding; decoding; combining; extracting; sampling; multiplexing; analog to digital converting; digital to analog converting; mathematically transforming; and any combination thereof.

74. The system of claim 1, wherein the guide assembly comprises a tissue contacting surface, and at least one of the guiding channels comprises a portion that is substantially parallel to at least a portion of the tissue contacting surface.

75. The system of claim 1, wherein the guide assembly comprises a tissue contacting surface, and at least one of the guiding channels comprises a portion that forms an approximately 45° angle with respect to at least a portion of the tissue contacting surface.

76. The system of claim 1, wherein at least one of the guiding channels or the corresponding one or more probes received in the at least one of the guiding channels comprises a conductive trace configured to provide an electrical connection between the at least one of the guiding channels and the one or more probes.

77. The system of claim 76, wherein energy is transferred via the electrical connection.

78. The system of claim 76, wherein signals are transferred via the electrical connection.

79. The system of claim 1, wherein at least one of the plurality of probes comprises one or more reservoirs or ports for delivery of an agent.

80. The system of claim 79, wherein the agent is a fluid.

81. The system of claim 79, wherein the probe assembly comprises a pump configured to supply the agent to the one or more reservoirs or ports.

82. The system of claim 81, wherein at least one of the one or more reservoirs or ports is refillable.

83. The system of claim 1, wherein the probe assembly is a micro electro-mechanical system.

84. The system of claim 83, wherein the micro electromechanical system is integrated into a silicon substrate.

85. The system of claim 1, wherein at least one of the plurality of probes comprises at least one electrode.

86. The system of claim 1, wherein at least one of the plurality of probes comprises at least one of: a recording electrode; a stimulating electrode; a sensor; an acoustic transducer; a light source; a heat source; a cooling source; an agent eluding port; and a reservoir.

87. The system of claim 1, wherein the guide assembly comprises a housing that defines the plurality of guiding channels, each of the guiding channels extending from an entry hole on a top surface of the housing to an exit hole on a tissue contacting surface.

88. The system of claim 87, wherein the tissue contacting surface is substantially opposite to the top surface.

89. The system of claim 87, wherein at least one of the guiding channels comprises an entry hole facing in a first axis on the top surface and an exit hole facing in a second axis on the tissue contacting surface, wherein the first axis and the second axis form an angle therebetween.

90. The system of claim 89, wherein the angle ranges from about 15° to about 90°.

91. The system of claim 87, wherein the tissue contacting surface comprises multiple planes.

92. The system of claim 87, wherein the exit holes on the tissue contacting surface are equally spaced.

93. The system of claim 87, wherein the entry holes on the top surface are arranged in a first pattern and the exit holes on the tissue contacting surface are arranged in a second pattern different from the first pattern.

94. The system of claim 87, wherein the entry holes on the top surface are arranged to receive the plurality of probes of the probe assembly.

95. The system of claim 87, wherein the guide assembly is custom made so that the tissue contacting surface of the housing closely matches the topography of a tissue surface of the patient to which the plurality of probes are to be placed.

96. The system of claim 87, wherein at least a portion of the tissue contacting surface is curved.

97. The system of claim 87, wherein at least a portion of the tissue contacting surface comprises a geometric shape selected from the group consisting of: a convex shape; a concave shape; a wedge shape; and a flat shape.

98. The system of claim 1, wherein at least one of the guiding channels is curved.

99. The system of claim 98, wherein the curved guiding channel is configured to guide a corresponding probe of the probe assembly along a predetermined tissue penetration trajectory.

100. The system of claim 1, wherein the probe assembly and the guide assembly are configured to engage one another.

101. The system of claim 100, wherein the probe assembly and the guide assembly engage one another via at least one of: a snap-fastener; a screw; a magnet; and a glue or adhesive.

102. The system of claim 100, wherein one of the probe assembly and the guide assembly comprises a projecting member, and the other of the probe assembly and the guide assembly comprises a corresponding hole to engage the projecting member.

103. The system of claim 100, wherein the engagement between the probe assembly and the guide assembly is one of a permanent engagement and a detachable engagement.

104. The system of claim 1, wherein one of the probe assembly and the guide assembly comprises a recess configured to receive the other of the probe assembly and the guide assembly.

105. The system of claim 1, further comprising a second probe assembly, wherein the guide assembly is configured to guide the probes of the second probe assembly.

106. The system of claim 1, further comprising a second guide assembly configured to guide the plurality of probes of the probe assembly.

107. The system of claim 1, wherein the guide assembly comprises a tubular housing.

108. The system of claim 107, wherein the tubular housing is formed from a flexible, substantially flat body.

109. The system of claim 108, wherein the flat body comprises a connecting member configured to connect ends of the flat body to form the tubular housing.

110. The system of claim 107, wherein the tubular housing is formed by two semi-circular portions connected together via a hinge.

111. The system of claim 107, wherein the tubular housing defines the plurality of guiding channels, at least one of the guiding channels extending from an entry hole on an outer surface of the tubular housing to an exit hole on an inner surface of the tubular housing.

112. The system of claim 1, further comprising a conduit for transmitting signals to an external device.

113. The system of claim 112, wherein the conduit comprises at least one of a wire and a fiber optic.

114. The system of claim 1 12, wherein the conduit is detachably connected to the probe assembly.

115. The system of claim 1, wherein the plurality of probes are sized and configured to penetrate tissue of the patient's central or peripheral nervous system.

116. The system of claim 1, wherein the plurality of probes are sized and configured to penetrate tumor tissue or organ tissue.

117. A device for guiding a plurality of probes, comprising:
a main body comprising:
a first surface having a plurality of first holes;
a second surface having a plurality of second holes; and
a plurality of guiding channels each extending between a respective first hole and a respective second hole, the guiding channels being configured to guide a plurality of probes to desired tissue sites.

118. The device of claim 117, wherein at least one of the plurality of guiding channels is curved.

119. The device of claim 117, wherein the second surface comprises a tissue contacting surface.

120. The device of claim 119, wherein at least a portion of the tissue contacting surface is custom made so that at least the portion of the tissue contacting surface closely matches the topography of the desired tissue site to which the plurality of probes are to be placed.

121. The device of claim 119, wherein at least a portion of the tissue contacting surface is curved.

122. The device of claim 119, wherein at least a portion of the tissue contacting surface comprises a geometric shape selected from the group consisting of: a convex shape; a concave shape; a wedge shape; and a flat shape.

123. The system of claim 1 17, wherein the first holes on the first surface are equally spaced.

124. The system of claim 117, wherein the second holes on the second surface are equally spaced.

125. The device of claim 117, wherein the first holes on the first surface are arranged in a first pattern and the second holes on the second surface are arranged in a second pattern different from the first pattern.

126. The device of claim 117, wherein the first holes on the first surface are arranged to receive the plurality of probes of a probe assembly.

127. The device of claim 117, wherein the main body is configured to engage with a probe assembly containing the plurality of probes.

128. The device of claim 117, wherein the main body is configured to engage with a plurality of probe assemblies each containing at least one probe.

129. The device of claim 117, wherein the main body has a tubular shape, the first surface being an outer surface of the main body, the second surface being an inner surface of the main body.

130. The device of claim 129, wherein the main body is formed from a flexible, substantially flat body.

131. The device of claim 130, wherein the flat body comprises a connecting member configured to connect ends of the flat body to form the main body.

132. The device of claim 129, wherein the main body is formed by two semi-circular portions, each semi-circular portion having a first end and a second end, the first ends pivotally connected to each other.

133. The device of claim 132, wherein the main body comprises a connecting member configured to connect the second ends of the two semi-circular portions together.

134. The device of claim 117, wherein the main body further comprises an anchor for attaching the main body to tissue near the desired tissue sites.

135. The device of claim 134, wherein the anchor comprises at least one tissue penetrating member.

136. The device of claim 135, wherein the anchor comprises at least two tissue penetrating members.

137. An electrode array comprising:
a housing; and
a plurality of probes extending from the housing,
wherein at least one of the plurality of probes is individually deployable from the housing.

138. The array of claim 137, wherein the at least one of the plurality of probes is retractable into the housing.

139. The array of claim 137, wherein at least two of the plurality of probes are simultaneously deployable from the housing.

140. The array of claim 137, wherein at least one of the probes is flexible.

141. The array of claim 137, wherein at least one of the probes is rigid.

142. The array of claim 137, wherein at least one of the probes has a resiliently biased shape.

143. The array of claim 142, wherein the resiliently biased shape has a curved portion.

144. The array of claim 137, wherein at least one of the probes comprises a shape memory material.

145. The array of claim 144, wherein the shape memory material comprises a shape memory alloy.

146. The array of claim 137, wherein at least two of the probes have lengths that are different from one another.

147. The array of claim 137, wherein at least two of the probes have thicknesses that are different from one another.

148. The array of claim 137, wherein at least one of the probes comprises a first functional element and a second functional element.

149. The array of claim 148, wherein the first functional element is different from the second functional element.

150. The array of claim 148, wherein at least one of the first and second functional elements comprises an electrode.

151. The array of claim 150, wherein the electrode is located at a distal tip of the probe.

152. The array of claim 148, wherein the first functional element is an electrode with a first set of characteristics, and the second functional element is an electrode with a second set of characteristics.

153. The array of claim 152, wherein the characteristics comprise at least one of: an impedance; a surface area; a material of construction; a surface texture; a porosity; a length; a width; a diameter; a thickness; a surface energy; a coating; and any combination thereof.

154. The array of claim 148, wherein at least one of the first and second functional elements comprises at least one of a photodiode and a photosensor.

155. The array of claim 147, wherein at least one of the probes comprises a conductive trace.

156. The array of claim 155, wherein the conductive trace is configured to mate with another trace disposed in the housing.

157. The array of claim 137, wherein at least one of the probes comprises a hollow lumen along at least a portion of its length.

158. The array of claim 137, wherein the plurality of probes are arranged in an array.

159. The array of claim 137, wherein the housing comprises a probe deployment mechanism configured to move one or more of the plurality of probes relative to the housing.

160. The array of claim 137, wherein at least one of the probes is configured to be deployed after the housing is implanted on a tissue surface of a patient.

161. The array of claim 137, further comprising a guide assembly comprising a plurality of guiding channels configured to guide one or more of the plurality of probes to a desired tissue site.

162. The array of claim 161, wherein the guide assembly comprises a tissue contacting surface, and at least one of the guiding channels comprises a portion that is substantially parallel to at least a portion of the tissue contacting surface.

163. The array of claim 161, wherein the guide assembly comprises a tissue contacting surface, and at least one of the guiding channels comprises a portion that forms an approximately 45° angle with respect to at least a portion of the tissue contacting surface.

164. The array of claim 161, wherein at least one of the guiding channels is curved.

165. The array of claim 164, wherein the curved guiding channel is configured to guide a corresponding probe along a predetermined tissue penetration trajectory.

166. The array of claim 161, wherein at least one of the guiding channels or the corresponding probe received in the at least one of the guiding channels comprises a trace configured to provide an electrical connection between the at least one of the guiding channels and the probe.

167. The array of claim 166, wherein energy is transferred via the electrical connection.

168. The array of claim 166, wherein signals are transferred via the electrical connection.

169. The array of claim 161, wherein at least one of the guiding channels comprises a first trace and the corresponding probe received in the at least one of the guiding channels comprises a second trace, the first trace and the second trace frictionally engage one another.

170. The array of claim 161, wherein the guide assembly comprises a tissue contacting surface, at least a portion of the tissue contacting surface is curved.

171. The array of claim 170, wherein the portion of the tissue contacting surface is custom made so that the tissue contacting surface closely matches the topography of a tissue surface to which the plurality of probes are to be placed.

172. The array of claim 161, wherein the guide assembly comprises a tissue contacting surface, at least a portion of the tissue contacting surface comprises a geometric shape selected from the group consisting of: a convex shape; a concave shape; a wedge shape; and a flat shape.

173. The array of claim 161, wherein the guide assembly comprises a guide housing that defines the plurality of guiding channels, each of the guiding channels extending from an entry hole on a top surface of the guide housing to an exit hole on a tissue contacting surface.

174. The array of claim 173, wherein the exit holes on the tissue contacting surface are arranged in an array.

175. The array of claim 174, wherein the plurality of guiding channels have at least 8 rows and at least 8 columns.

176. The array of claim 173, wherein the exit holes on the tissue contacting surface are equally spaced.

177. The array of claim 161, wherein the guide assembly comprises a tubular housing defining the plurality of guiding channels.

178. The array of claim 177, wherein at least one of the guiding channels extends from an entry hole on an outer surface of the tubular housing to an exit hole on an inner surface of the tubular housing.

179. The array of claim 138, further comprising a conduit for transmitting signals to an external device.

180. The array of claim 179, wherein the conduit comprises at least one of a wire and a fiber optic.

181. The array of claim 179, wherein the conduit is detachably connected to the housing.

182. The array of claim 138, wherein the housing comprises at least one internal guiding lumen configured to receive one or more probes, the housing comprising a drive assembly positioned adjacent the internal guiding lumen and being configured to move one or more probes along the internal guiding lumen.

183. The array of claim 182, wherein the drive assembly is manually controllable.

184. The array of claim 182, wherein the drive assembly is remotely controllable.

185. The array of claim 182, wherein the drive assembly is automatically controllable.

186. The array of claim 185, wherein the plurality of probes comprises a signal detector, wherein at least one of the plurality of probes is configured to move when a quality of a signal detected by the signal detector falls below a threshold level.

187. The array of claim 186, wherein the signal detected by the signal detector comprises signals used in diagnosis of: obesity; an eating disorder; a neurological disorder; a stroke; a coma; amnesia; irregular blood flow in the brain; a psychiatric disorder; a cardiovascular disorder; an endocrine disorder; sexual dysfunction; incontinence; a hearing disorder; a visual disorder; a sleeping disorder; a movement disorder; impaired limb function; absence of a limb or a limb portion; a speech disorder; a physical injury; migraine headaches; stroke; a chronic or severe pain condition; or any combination thereof.

188. The array of claim 185, wherein at least one of the plurality of probes is configured to transmit a therapy signal, wherein at least one of the plurality of probes is configured to move when a quality of the therapy signal falls below a threshold level.

189. The array of claim 188, wherein the therapy signal comprises signals used in treatment of: obesity; an eating disorder; a neurological disorder; a stroke; a coma; amnesia; irregular blood flow in the brain; a psychiatric disorder; a cardiovascular disorder; an endocrine disorder; sexual dysfunction; incontinence; a hearing disorder; a visual disorder; a sleeping disorder; a movement disorder; impaired limb function; absence of a limb or a limb portion; a speech disorder; a physical injury; migraine headaches; stroke; a chronic or severe pain condition; or any combination thereof.

190. The array of claim 182, wherein the drive assembly comprises:
a screw extending along at least a portion of the internal guiding lumen,
a drive member configured to engage the one or more probes and the screw; and
a drive mechanism configured to drive the drive member so as to move the one or more probes along the internal guiding lumen.

191. The array of claim 182, wherein the drive assembly comprises at least one pinch roller in contact with the one or more probes, wherein rotating the roller causes the one or more probes to move distally or proximally along the internal guiding lumen.

192. The array of claim 191, wherein the at least one pinch roller is disposed adjacent the internal guiding lumen or has a portion disposed in the internal guiding lumen.

193. The array of claim 191, wherein the at least one pinch roller comprises two pinch rollers.

194. The array of claim 182, wherein the drive assembly comprises a gas discharging member having an outlet valve and being configured to discharge gas into the internal guiding lumen, wherein discharge of the gas causes the one or more probes to advance the probe distally along the internal guiding lumen.

195. The array of claim 194, wherein the gas discharging member comprises an electrolytic cell.

196. The array of claim 194, wherein the drive assembly further comprises a gas suction member having an inlet valve and being configured to suction gas out of the internal guiding lumen, wherein suctioning of the gas causes the one or more probes to retract proximally along the internal guiding lumen.

197. The array of claim 182, wherein the drive assembly comprises a suction member having an inlet valve and being configured to suction fluid out of the internal guiding lumen so as to retract the one or more probes proximally along the internal guiding lumen.

198. The array of claim 182, wherein the drive assembly comprises:
an extendable piston having a distal end connected to the probe; and
a drive mechanism configured to extend or retract the extendable piston so as to move the probe distally or proximally along the internal guiding lumen.

199. The array of claim 198, wherein the drive mechanism comprises at least one of a hydraulic drive element and a pneumatic drive element.

200. The array of claim 182, wherein the drive assembly comprises:
a roller coupled to a proximal end of the one or more probes, a surface of the roller being in contact with an inner surface of the internal guiding lumen; and
a controller configured to control rotation of the roller,
wherein rotation of the roller causes the one or more probes to move distally or proximally along the internal guiding lumen.

201. The array of claim 200, wherein the drive assembly further comprises a second roller coupled to the one or more probes.

202. The array of claim 182, wherein the drive assembly comprises:
a tube having inner threads and being disposed inside the internal guiding lumen;
a screw attached to a proximal end of the one or more probes, the screw being configured to engage with and ride over the inner threads; and
a drive mechanism configured to rotate at least one of the tube and the screw,
wherein rotating at least one of the tube and the screw causes the screw to move relative to the tube so as to move the one or more probes distally or proximally along the internal guiding lumen.

203. The array of claim 202, wherein the drive mechanism comprises a stepper motor.

204. The array of claim 182, wherein the drive assembly comprises:
first teeth disposed at least partially along the internal guiding lumen;
a drive member coupled to the one or more probes, the drive member comprising a forward-moving member configured to engage or disengage at least one tooth of the first teeth when a first predetermined condition is applied, such that when the forward-moving member engages and disengages the at least one tooth of the first teeth, the probe moves distally along the internal guiding lumen; and
an actuator configured to actuate the forward-moving member so as to move the probe distally along the internal guiding lumen.

205. The array of claim 204, wherein the forward-moving member comprises a shape memory material.

206. The array of claim 204, wherein the forward-moving member is disengaged from the at least one tooth of the first teeth when the one or more probes moves proximally.

207. The array of claim 204, wherein the drive assembly further comprises second teeth disposed at least partially along the internal guiding lumen, and wherein the drive member further comprises a backward-moving member configured to engage or disengage at least one tooth of the second teeth when a second predetermined condition is applied, so that when the backward-moving member engages and disengages the at least one tooth of the second teeth, the one or more probes moves proximally along the internal guiding lumen.

208. The array of claim 182, wherein the drive assembly comprises:
teeth disposed at least partially along the internal guiding lumen;
a drive member coupled to the probe, the drive member comprising a backward-moving member configured to engage or disengage at least one tooth when a first predetermined condition is applied, such that when the backward-moving member engages and disengages the at least one tooth, the one or more probes moves proximally along the internal guiding lumen; and
an actuator configured to actuate the backward-moving member so as to move the one or more probes proximally along the internal guiding lumen.

209. The array of claim 208, wherein the backward-moving member comprises a shape memory material.

210. The array of claim 208, wherein the backward-moving member is disengaged from the at least one tooth when the one or more probes moves distally.

211. The array of claim 181, wherein the drive assembly comprises:
at least one first magnet disposed at least partially along the internal guiding lumen;
a drive member coupled to the probe and comprising at least one second magnet; and
a controller for energizing either the first magnet or the second magnet,
wherein energizing either the first magnet or the second magnet causes the one or more probes to move distally or proximally along the internal guiding lumen.

212. The array of claim 211, wherein at least one of the first and second magnets is configured to be activated by the controller.

213. The array of claim 211, wherein at least one of the first and second magnets is an electromagnet.

214. The array of claim 211, wherein at least one of the first and second magnets is a permanent magnet.

215. The array of claim 211, wherein one of the first and second magnets is an electromagnet, and the other of the first and second magnets is a permanent magnet.

216. The array of claim 211, wherein at least one of the first and second magnets comprises a plurality of magnets.

217. The array of claim 211, wherein the controller is configured to energize either the first magnet or the second magnet by supplying electrical current to the magnet to be energized.

218. The array of claim 217, wherein the electrical current supplied in a first direction causes the one or more probes to move distally along the internal guiding lumen, and the electrical current supplied in a second direction opposite the first direction causes the one or more probes to move proximally along the internal guiding lumen.

219. The array of claim 211, wherein the at least one first magnet or the at least one second magnet comprises a plurality of magnets arranged in a row, the plurality of magnets separated from one another by a predetermined distance.

220. The array of claim 219, wherein energizing either the first magnet or the second magnet causes the one or more probes to move a length that is substantially equal to the predetermined distance.

221. The array of claim 138, wherein the housing further comprises at least one of: a memory storage device; a signal processing unit; a power transfer device; a power conversion device; a wireless communication device; a CPU; a microcontroller; a drug delivery assembly or reservoir; an electromagnetic field generator; a light source; a camera assembly; an impedance measurement device; a radiopaque marker; and a power supply.

222. The array of claim 138, wherein the housing further comprises a power transfer device configured to convert non-electrical energy to electrical energy.

223. The array of claim 138, wherein the housing comprises a wireless communication device configured to transfer information via: radiofrequency; microwave; infrared; ultrasound; or any combination thereof.

224. The array of claim 138, wherein the housing further comprises a signal processing element configured to perform a signal processing function selected from the group consisting of: amplification; filtering; sorting; conditioning; translating; interpreting; encoding; decoding; combining; extracting; sampling; multiplexing; analog to digital converting; digital to analog converting; mathematically transforming; and any combination thereof.

225. The array of claim 138, wherein at least one of the plurality of probes comprises one or more reservoirs or ports for delivery of an agent.

226. The array of claim 225, wherein the agent is a fluid.

227. The array of claim 225, further comprising s a pump configured to supply the agent to the one or more reservoirs or ports.

228. The array of claim 227, wherein at least one of the one or more reservoirs or ports is refillable.

229. The array of claim 138, wherein the housing and the plurality of probes are a micro electromechanical system.

230. The array of claim 229, wherein the micro electromechanical system is integrated into a silicon substrate.

231. The array of claim 138, wherein at least one of the plurality of probes comprises at least one electrode.

232. The array of claim 138, wherein at least one of the plurality of probes comprises at least one of: a recording electrode; a stimulating electrode; a sensor; an acoustic transducer; a light source; a heat source; a cooling source; an agent eluding port; and a reservoir.

233. The array of claim 138, further comprising an anchor for anchoring the array to a tissue surface to which the plurality of probes are to be inserted.

234. The array of claim 233, wherein the anchor comprises at least one tissue penetrating member.

235. The array of claim 233, wherein the anchor comprises at least two tissue penetrating members.

236. A kit used for implanting an electrode system, comprising:
a probe assembly comprising a plurality of probes configured to penetrate tissue of a patient; and
a first guide assembly and a second guide assembly, each of the first and second guide assemblies comprising a housing defining a plurality of guiding channels, each of the guiding channels extending between an entry hole on a first surface of the housing and an exit hole on a second surface of the housing,
wherein the entry holes of the first guide assembly and the entry holes of the second guide assembly are arranged in substantially identical patterns, and
wherein the second surface of the first guide assembly has a characteristic differing from that of the second surface of the second guide assembly.

237. The kit of claim 236, wherein each of the first surfaces of the first and second guide assemblies is configured to engage with the probe assembly.

238. The kit of claim 237, wherein the entry holes on each of the first surfaces of the first and second guide assemblies is arranged such that, when the probe assembly engages with one of the first and second guide assemblies, the plurality of probes are inserted into the entry holes.

239. The kit of claim 236, wherein the characteristic is a contour of the second surface of the first guide assembly that is different from a contour of the second surface of the second guide assembly.

240. The kit of claim 236, wherein the characteristic is an arrangement of the exit holes on the second surface of the first guide assembly that is different from an arrangement of the exit holes on the second surface of the second guide assembly.

241. The kit of claim 236, wherein the first and second guide assemblies are configured such that each of the second surfaces are contoured to substantially match a different tissue surface of a patient.

242. The kit of claim 236, wherein the first and second guide assemblies are custom made so that second surfaces are contoured to substantially match a different tissue surface of a particular patient.

243. The kit of claim 236, wherein at least one of the plurality of guiding channels in at least one of the first and second guide assemblies is curved.

244. The kit of claim 236, wherein at least one of the first and second guide assemblies comprises a recess configured to receive the probe assembly.

245. The kit of claim 236, wherein the plurality of guiding channels in at least one of the first and second guide assemblies are configured to guide the probes in different penetration trajectories.

246. The kit of claim 236, wherein the probe assembly and the guide assembly are configured to engage one another.

247. The kit of claim 246, wherein the probe assembly and the guide assembly engage one another via at least one of: a snap-fastener; a screw; a magnet; and a glue or adhesive.

248. The kit of claim 246, wherein one of the probe assembly and the guide assembly comprises a projecting member, and the other of the probe assembly and the guide assembly comprises a corresponding hole to engage the projecting member.

249. The kit of claim 246, wherein the engagement between the probe assembly and the guide assembly is one of a permanent engagement and a detachable engagement.

250. The kit of claim 236, further comprising a signal processing element configured to perform a signal processing function selected from the group consisting of: amplification; filtering; sorting; conditioning; translating; interpreting; encoding; decoding; combining; extracting; sampling; multiplexing; analog to digital converting; digital to analog converting; mathematically transforming; and any combination thereof.

251. The kit of claim 250, further comprising a communication device configured to send and/or receive signals from and/or to the signal processing element.

252. The kit of claim 236, further comprising at least one of a therapeutic device or a diagnostic device configured to communicate with the communication device.

253. A method of inserting a probe assembly into a patient, comprising:
providing a kit of claim 235;
determining a topography of a tissue surface into which the probe assembly is to be inserted;
selecting at least one of the first and second guide assemblies that closely matches the topography of the tissue surface;
placing the selected guide assembly onto the tissue surface with the second surface in contact with the tissue surface; and
inserting the plurality of probes into the entry holes on the first surface.

254. The method of claim 253, wherein at least one of the guide assemblies is custom made to match the topography.

255. The method of claim 253, wherein determining the topography comprises performing at least one of: a magnetic resonance imaging (MRI), a functional MRI, a computed tomography (CT-scan), an ultrasound imaging procedure, an X-ray imaging, or a fluoroscopy.

256. The method of claim 255, wherein placing the selected guide assembly comprises anchoring the selected guide assembly on the tissue surface prior to inserting the plurality of probes.

257. A method of implanting a plurality of probes into a patient, comprising:
providing a plurality of probes;
determining a topography of a tissue surface into which the probes are to be inserted;
providing a guide assembly comprising:
a first surface having a plurality of entry holes configured to receive the plurality of probes;
a second surface having a plurality of exit holes, the second surface having a surface contour substantially matching the topography of the tissue surface; and
a plurality of guiding channels each extending from a corresponding entry hole on the first surface to a corresponding exit hole on the second surface;
bringing the second surface of the guide assembly in contact with the tissue surface; and
inserting at least one of the probes into the entry holes of the guide assembly.

258. The method of claim 257, wherein the plurality of probes are arranged in one or more probe assemblies.

259. The method of claim 258, wherein one of the probe assembly and the guide assembly comprises a recess configured to receive the other of the probe assembly and the guide assembly.

260. The method of claim 257, wherein the guide assembly is custom made so that at least the second surface of the guide assembly substantially matches the topography of the tissue surface.

261. The method of claim 260, wherein the guide assembly is custom made by utilizing at least one of: a magnetic resonance imaging (MRI); a functional MRI; a computed tomography (CT-scan); an ultrasound imaging; an X-ray imaging; and a fluoroscope.

262. The method of claim 257, wherein determining the topography comprises performing at least one of: a magnetic resonance imaging (MRI); a functional MRI; a computed tomography (CT-scan); an ultrasound imaging; an X-ray imaging; and a fluoroscopy.

263. The method of claim 257, wherein at least one of the plurality of guiding channels is curved.

264. The method of claim 257, wherein the plurality of guiding channels are configured to guide the probes in different penetration trajectories.

265. The method of claim 257, wherein the plurality of probes are arranged in a housing, and at least one of the plurality of probes is individually deployable from the housing.

266. The method of claim 265, wherein the housing comprises a probe deployment mechanism configured to move the at least one of the plurality of probes relative to the housing.

267. The method of claim 257, wherein the entry holes on the first surface are arranged in a first pattern and the exit holes on the second surface are arranged in a second pattern, different from the first pattern.

268. The method of claim 257, wherein at least one of the probes is movable relative to another of the probes.

269. The method of claim 257, further comprising moving at least one of the probes relative to another of the probes after inserting the probes into the entry holes of the guide assembly.

270. The method of claim 257, further comprising moving at least two of the probes simultaneously after inserting the probes into the entry holes of the guide assembly.

271. The method of claim 257, wherein bringing the second surface of the guide assembly in contact with the tissue surface comprises anchoring the guide assembly on the tissue surface.

272. The method of claim 271, wherein anchoring the guide assembly on the tissue surface is performed prior to inserting the at least one of the probes into the entry holes.

273. A method of implanting an electrode sensor system, comprising:
providing an electrode system comprising at least one probe, a processing unit, and a conduit for transmitting signals between the probe and the processing unit;
creating an opening in the skull;
inserting the probe through the opening;
placing the processing unit on an external portion of the skull;
creating a slot on the surface of the skull, the slot extending at least partially from the opening to the processing unit; and
placing the conduit in the slot.

274. The method of claim 273, wherein inserting the probe through the opening comprises inserting the probe at least partially into the brain.

275. The method of claim 273, wherein the at least one probe comprises a plurality of probes.

276. The method of claim 275, wherein at least one of the plurality of probes comprises at least one of: a recording electrode; a stimulating electrode; a sensor; an acoustic transducer; a light source; a heat source; a cooling source; an agent eluding port; and a reservoir.

277. The method of claim 275, wherein at least one of the plurality of probes comprises at least one electrode.

278. The method of claim 275, further comprising connecting the conduit to one or more additional probes.

279. The method of claim 273, wherein the probe is configured to record cellular activity.

280. The method of claim 273, wherein the probe is configured to deliver energy to tissue.

281. The method of claim 280, wherein the energy delivered comprises at least one selected from the group consisting of: heat energy; cryogenic energy; light energy; radiation energy; chemical energy; mechanical energy; electrical energy; and any combination thereof.

282. The method of claim 273, wherein the probe is configured to deliver agent.

283. The method of claim 282, wherein the agent comprises a pharmaceutical agent.

284. The method of claim 273, wherein the probe comprises a sensor.

285. The method of claim 284, wherein the sensor comprises at least one selected from the group consisting of: a thermal sensor; a pressure sensor; a chemical sensor; a force sensor; an electromagnetic field sensor; a physiologic sensor; a photodetector; a pH sensor; an oxygen sensor; a blood sensor; an electrode; and any combination thereof.

286. The method of claim 273, wherein the processing unit is located less than 20 cm from the probe.

287. The method of claim 273, further comprising placing the processing unit above the skin of the patient.

288. The method of claim 273, further comprising placing the processing unit on top of the skull of the patient under the scalp.

289. A method of implanting a plurality of probes into a patient, comprising:
providing a probe assembly having a main body and a plurality of probes extending from the main body, at least one of the plurality of probes being movable relative to the main body;
inserting the plurality of probes into tissue of the patient;
detecting signals with the at least one of the plurality of probes; and
selectively moving the at least one of the plurality of probes relative to the main body until the at least one of the plurality of probes detects signals having a desired signal strength.

290. The method of claim 289, wherein selectively moving is controlled automatically.

291. The method of claim 289, wherein selectively moving is controlled manually.

292. The method of claim 289, wherein selectively moving is controlled remotely.

293. The method of claim 289, wherein selectively moving comprises advancing or retracting the at least one of the plurality of probes.

294. The method of claim 289, further comprising transmitting stimulating signals into the tissue.

295. The method of claim 294, wherein detecting signals comprises detecting signals from the tissue responsive to the stimulating signals.

296. The method of claim 289, wherein selectively moving the at least one of the plurality of probes is performed after the step of inserting the plurality of probes into tissue of the patient.

297. The method of claim 289, wherein the desired signal strength is above a predetermined threshold level.

298. The method of claim 297, further comprising adjusting the predetermined threshold level.

299. A method of implanting a plurality of probes into a patient, comprising:
providing a probe assembly having a main body and a plurality of probes extending from the main body, at least one of the plurality of probes being movable relative to the main body;
inserting the plurality of probes into tissue of the patient;
transmitting therapeutic signals to the tissue with the at least one of the plurality of probes; and
selectively moving the at least one of the plurality of probes relative to the main body until a desired therapeutic result is achieved.

300. The method of claim 299, wherein the desired therapeutic result comprises at least one of: prevention or reduction of a seizure; reduction in pain; improvement in cellular activity; or improvement in motor function of a patient, in response to the therapeutic signals.

301. The method of claim 299, further comprising observing the patient's condition relating to the desired therapeutic result.

302. The method of claim 301, wherein the observing is performed with at least one sensor selected from the group consisting of: a thermal sensor; a pressure sensor; a chemical sensor; a force sensor; an electromagnetic field sensor; a physiologic sensor; a photodetector; a pH sensor; an oxygen sensor; a blood sensor; and any combination thereof.

303. The method of claim 301, further comprising stopping the selective movement of the at least one of the plurality of probes when a change in the patient's condition exceeds a predetermined threshold level.

304. The method of claim 301, wherein the observing is performed by a visual observation of the patient.

305. The method of claim 299, wherein selectively moving is controlled automatically.

306. The method of claim 299, wherein selectively moving is controlled manually.

307. The method of claim 299, wherein selectively moving is controlled remotely.

308. The method of claim 299, wherein selectively moving comprises advancing or retracting the at least one of the plurality of probes.

309. The method of claim 299, further comprising transmitting stimulating signals into the tissue.

310. The method of claim 309, wherein detecting signals comprises detecting signals from the tissue responsive to the stimulating signals.

311. The method of claim 299, wherein selectively moving the at least one of the plurality of probes is performed after the step of inserting the plurality of probes into tissue of the patient.

312. The method of claim 299, wherein the desired therapeutic result is above a predetermined threshold level.

313. The method of claim 312, further comprising adjusting the predetermined threshold level.

314. A system comprising:
the electrode system of claim 1; and
a functional device associated with the electrode system.

315. The system of claim 314, wherein at least one of the plurality of probes comprises a sensor configured to detect signals generated from one or more living cells, and the functional device is controllable by a control signal generated based on the signals detected by the sensor.

316. The system of claim 315, further comprising a processing unit configured to receive the detected signals to produce processed signals.

317. The system of claim 316, wherein the processing unit receives the detected signals wirelessly.

318. The system of claim 316, wherein the processed signals comprise the control signal.

319. The system of claim 318, wherein the processing unit is configured to transmit the control signal to the functional device wirelessly.

320. The system of claim 316, wherein the processing unit is implanted in the patient's body.

321. The system of claim 316, wherein the processing unit is placed external to the patent's body.

322. The system of claim 316, wherein the processing unit is configured to perform at least one of: amplification; filtering; sorting; conditioning; translating; interpreting; encoding; decoding; combining; extracting; sampling; multiplexing; analog to digital converting; digital to analog converting; and mathematically transforming.

323. The system of claim 314, wherein the functional device is controlled by control signals generated under voluntary control of the patient.

324. The system of claim 314, wherein the functional device is configured to receive wireless signals from the probe system.

325. The system of claim 314, wherein at least one of the plurality of probes is configured to send signals to one or more living cells.

326. The system of claim 325, wherein the functional device is configured to transmit the signals to the at least one of the plurality of probes.

327. The system of claim 325, further comprising a processing unit configured to transmit the signals to the at least one of the plurality of probes.

328. The system of claim 327, wherein the processing unit is configured to perform at least one of: amplification; filtering; sorting; conditioning; translating; interpreting; encoding; decoding; combining; extracting; sampling; multiplexing; analog to digital converting; digital to analog converting; and mathematically transforming.

329. The system of claim 325, wherein the signals are configured to polarize, stimulate, or affect the one or more living cells.

330. The system of claim 325, wherein the signals comprise at least one of: electric current, an electromagnetic field, acoustic energy, heat energy, cooling energy, pharmaceutical drug or agent, light, and mechanical vibration.

331. The system of claim 314, wherein the functional device comprises at least one of:
a therapeutic device; a restorative device; and diagnostic device.

332. The system of claim 331, wherein the therapeutic device is configured to perform a therapeutic function comprising a treatment of one or more of: obesity, an eating disorder, a neurological disorder, a psychiatric disorder, a cardiovascular disorder, an endocrine disorder, sexual dysfunction, incontinence, a hearing disorder, a visual disorder, a sleeping disorder, a movement disorder, a speech disorder, physical injury, migraine headaches, stroke, and chronic pain.

333. The system of claim 331, wherein the diagnostic device is configured to perform a patient diagnosis comprising a diagnosis of one or more of: obesity, an eating disorder, a neurological disorder, a psychiatric disorder, a cardiovascular disorder, an endocrine disorder, sexual dysfunction, incontinence, a hearing disorder, a visual disorder, sleeping disorder, a movement disorder, a speech disorder, physical injury, migraine headaches, stroke, and chronic pain.

334. The system of claim 331, wherein the restorative device is configured to restore a bodily function of the patient, the bodily function comprising one or more of vision, hearing, speech, communication, limb motion, ambulation, reaching, grasping, standing, rolling over, bowel movement, and bladder evacuation.

335. The system of claim 314, wherein the functional device is implanted in the patient's body.

336. The system of claim 314, wherein the functional device is placed external to the patent's body.

337. The system of claim 314, wherein the functional device comprises at least one selected from the group consisting of: a computer, a computer display, a mouse, a cursor, a joystick, a personal data assistant, a robot or robotic component, a computer controlled device, a teleoperated device, a communication device, a vehicle, an adjustable bed, an adjustable chair, a remote controlled device, a Functional Electrical Stimulator device, a muscle stimulator, an exoskeletal robot brace, an artificial or prosthetic limb, a vision enhancing device, a vision restoring device, a hearing enhancing device, a hearing restoring device, a movement assist device, a medical therapeutic equipment, a drug delivery apparatus, a medical diagnostic equipment, a bladder control device, a bowel control device, a human enhancement device, and a closed loop medical equipment.

338. A system comprising:
an electrode array of claim 137; and
a functional device associated with the electrode array.

339. The system of claim 338, wherein at least one of the plurality of probes comprises a sensor configured to detect signals generated from one or more living cells, and the functional device is controllable by a control signal generated based on the signals detected by the sensor.

340. The system of claim 339, further comprising a processing unit configured to receive the detected signals to produce processed signals.

341. The system of claim 340, wherein the processing unit receives the detected signals wirelessly.

342. The system of claim 340, wherein the processed signals comprise the control signal.

343. The system of claim 342, wherein the processing unit is configured to transmit the control signal to the functional device wirelessly.

344. The system of claim 340, wherein the processing unit is implanted in the patient's body.

345. The system of claim 340, wherein the processing unit is placed external to the patient's body.

346. The system of claim 340, wherein the processing unit is configured to perform at least one of: amplification; filtering; sorting; conditioning; translating; interpreting; encoding; decoding; combining; extracting; sampling; multiplexing; analog to digital converting; digital to analog converting; and mathematically transforming.

347. The system of claim 338, wherein the functional device is controlled by control signals generated under voluntary control of the patient.

348. The system of claim 338, wherein the functional device is configured to receive wireless signals from the electrode array.

349. The system of claim 338, wherein at least one of the plurality of probes is configured to send signals to one or more living cells.

350. The system of claim 349, wherein the functional device is configured to transmit the signals to the at least one of the plurality of probes.

351. The system of claim 349, further comprising a processing unit configured to transmit the signals to the at least one of the plurality of probes.

352. The system of claim 351, wherein the processing unit is configured to perform at least one of: amplification; filtering; sorting; conditioning; translating; interpreting; encoding; decoding; combining; extracting; sampling; multiplexing; analog to digital converting; digital to analog converting; and mathematically transforming.

353. The system of claim 349, wherein the signals are configured to polarize, stimulate, or affect one or more living cells adjacent the at least one of the plurality of probes.

354. The system of claim 349, wherein the signals comprise at least one of: electric current, an electromagnetic field, acoustic energy, heat energy, cooling energy, pharmaceutical drug or agent, light, and mechanical vibration.

355. The system of claim 338, wherein the functional device comprises at least one of:
a therapeutic device; a restorative device; and diagnostic device.

356. The system of claim 355, wherein the therapeutic device is configured to perform a therapeutic function comprising a treatment of one or more of: obesity, an eating disorder, a neurological disorder, a psychiatric disorder, a cardiovascular disorder, an endocrine disorder, sexual dysfunction, incontinence, a hearing disorder, a visual disorder, a sleeping disorder, a movement disorder, a speech disorder, physical injury, migraine headaches, stroke, and chronic pain.

357. The system of claim 355, wherein the diagnostic device is configured to perform a patient diagnosis comprising a diagnosis of one or more of: obesity, an eating disorder, a neurological disorder, a psychiatric disorder, a cardiovascular disorder, an endocrine disorder, sexual dysfunction, incontinence, a hearing disorder, a visual disorder, sleeping disorder, a movement disorder, a speech disorder, physical injury, migraine headaches, stroke, and chronic pain.

358. The system of claim 355, wherein the restorative device is configured to restore a bodily function of the patient, the bodily function comprising one or more of vision, hearing, speech, communication, limb motion, ambulation, reaching, grasping, standing, rolling over, bowel movement, and bladder evacuation.

359. The system of claim 338, wherein the functional device is implanted in a patient's body.

360. The system of claim 338, wherein the functional device is placed external to a patent's body.

361. The system of claim 338, wherein the functional device comprises at least one selected from the group consisting of: a computer, a computer display, a mouse, a cursor, a joystick, a personal data assistant, a robot or robotic component, a computer controlled device, a teleoperated device, a communication device, a vehicle, an adjustable bed, an adjustable chair, a remote controlled device, a Functional Electrical Stimulator device, a muscle stimulator, an exoskeletal robot brace, an artificial or prosthetic limb, a vision enhancing device, a vision restoring device, a hearing enhancing device, a hearing restoring device, a movement assist device, a medical therapeutic equipment, a drug delivery apparatus, a medical diagnostic equipment, a bladder control device, a bowel control device, a human enhancement device, and a closed loop medical equipment.
